(19) United States
(12) Patent Application Publication
(10) Pub. No.: US 2009/0018169 A1
(43) Pub. Date: Jan. 15, 2009

(54) Sulfonamide Derivatives for the Treatment of Bacterial Infections

(75) Inventors: Jerome Gonzalez, Annemasse (FR); Dominique Swinnen, Beaumont (FR); Agnes Bombrun, Chambesy (CH); Rob Hooft Van Huijsduijnen, Bernex (CH); Timothy Wells, Chambesy (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Laboratoires Serono SA, Coinsins,Vaud (CH)

(21) Appl. No.: 12/278,831

(22) PCT Filed: Feb. 9, 2007
(86) PCT No.: PCT/EP07/51242

§ 371 Date: Aug. 8, 2008

Related U.S. Application Data
(60) Provisional application No. 60/772,793, filed on Feb. 13, 2006.

(30) Foreign Application Priority Data
Feb. 13, 2006 (EP) 06101574.9

(51) Int. Cl. A61K 31/381; C07D 409/12; A61K 31/4436; A61P 31/04; C07D 333/04

(52) U.S. Cl. 514/336; 546/280.4; 549/65; 514/445

(57) ABSTRACT

This present invention is related to sulfonamide derivatives of Formula (I), pharmaceutical composition thereof, methods of preparation thereof and to their use for the treatment and/or prophylaxis of bacterial infections such as tuberculosis.

### FIELD OF THE INVENTION

This present invention is related to sulfonamide derivatives of Formula (I), pharmaceutical composition thereof, methods of preparation thereof and to their use for the treatment and/or prophylaxis of bacterial infections such as tuberculosis. Specifically, the present invention is related to sulfonamide derivatives for the modulation, notably the inhibition of the activity or function of the Mycobacterium tuberculosis Protein Tyrosine Phosphatases (MPTP), especially MPTPB.

### BACKGROUND OF THE INVENTION

Tuberculosis (TB) is a contagious bacterial infection caused by Mycobacterium tuberculosis. Although the lungs are primarily involved, infection can spread to other organs. According to the World Health Organization (WHO), one-third of the world population is infected with tubercle bacilli (Mycobacterium tuberculosis) that causes 5,000 people to die of TB daily (Doggrell, 2005, Expert Opin. Invest. Drugs, 14(7), 917-920); about 35 million people are expected to die from TB in the first 20 years of the 21^{st} century. The main reasons for this are: the spread of HIV/AIDS, the lack of compliance with present treatments, the variable efficacy of the Bacille Calmette-Guerin (BCG) vaccine and the emergence of multi drug-resistant TB strains (MDRTB). HIV infection is the most potent risk factor for converting latent TB into active transmissible TB. One third of AIDS patients have TB and one third of mortalities associated with AIDS are caused by TB.

Pathogenicity of a microorganism normally depends on the ability of the organism to survive and replicate in the host. During infection, M. tuberculosis resides primarily within macrophages and is exposed to many adverse conditions, including hypoxia reactive nitrogen or oxygen species, nutritional deprivation and other macrophage bactericidal systems produced during immune surveillance. The ability of M. tuberculosis to enter macrophages and to circumvent host immune response mechanisms in order to enhance their intracellular survival is believed to be crucial for its virulence.

It has been found that M. tuberculosis has two functional secretory tyrosine phosphatases (low molecular weight phosphatases), namely MptpA and MptpB (Koul et al., 2000, J. Bacteriol., 182, 5425-5432). MptpB is optimally active at pH 5.6, similar to the pH of the lysosomal compartment, and is present exclusively in pathogenic strains of M. tuberculosis complex. The MptpB mutant strain shows impaired ability to survive in activated macrophages in guinea pig but not in resting macrophages, suggesting its importance in the host-pathogen interaction (Singh et al., 2003, Mol. Microbiol., 50, 751-762).

WO 2005/005639 provides mutant Mycobacterium strains harboring a modified tyrosine phosphatase gene (MptpA and/or MptpB) wherein the mutant Mycobacterium strain is said to be incapable of expressing the active tyrosine phosphatase and proposes a method to assess the role of tyrosine phosphatases MptpA and MptpB in the virulence and pathogenesis of Mycobacterium which can be used as potential targets for developing anti-tubercular drug.

Therefore, it has been suggested that the inhibition of secretory tyrosine phosphatases from mycobacteria (e.g. MptpA and/or MptpB), would inhibit or prevent mycobacterial growth (WO 01/81422) and therefore would be beneficial in the treatment of TB. Therefore, it is believed that the development of inhibitors of microbacterial phosphatases, would be beneficial in the treatment of TB.

Isoniazid and rifampin were developed in the 1950s and 1960s and there were not major new drugs in the last 40 years.

Standard treatments of TB, as recommended by the WHO, lasts at least 6 months and requires a combination of different antibiotics (Van Daele et al., 2005, Expert Opin. Ther. Patents, 15(2), 131-140)

Recently, antimycobacterial small molecule therapeutics have been developed as described in Van Daele et al., 2005, above, D O Davies et al., 2003, Expert Opin. Invest. Drugs, 12(8), 1297-1312 and Doggrell, 2005, above and in Tangallapally et al., 2005, J. Med. Chem., 2005, 48(26), 8261-8269.

The high relevance of the low molecular weight phosphatase pathway in some widely spread bacterial infections, stresses the need to develop new potent drugs for TB.

### SUMMARY OF THE INVENTION

It is an object of the invention to provide substances which are suitable for the treatment and/or prevention of bacterial infections such as tuberculosis and diseases that result from immunosupression by tuberculosis such as pneumonia and AIDS.

It is notably an object of the present invention to provide chemical compounds which are able to modulate, especially inhibit the activity or function of secretory tyrosine phosphatases from mycobacteria (e.g. MptpA and/or MptpB), especially MptpB.

It is furthermore an object of the present invention to provide a new category of pharmaceutical formulations for the treatment of and/or bacterial infections such as tuberculosis and diseases that result from immunosupression by tuberculosis such as pneumonia and AIDS.

It is furthermore an object of the present invention to provide a method for the treatment and/or prevention of bacterial infections such as tuberculosis and diseases that result from immunosupression by tuberculosis such as pneumonia and AIDS.

In a first aspect, the invention provides the use of oxamic acid derivatives of Formula (I):

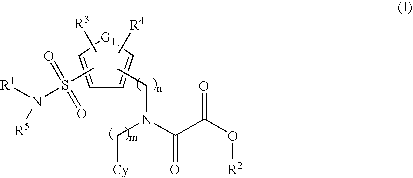

wherein G¹, R¹, R², R³, R⁴, R⁵, Cy, m and n are defined in the detailed description below for the preparation of a pharmaceutical formulation for the prevention and/or treatment of bacterial infections such as tuberculosis and diseases that result from immunosupression by tuberculosis such as pneumonia and AIDS.

In a second aspect, the invention provides the use of sulfonamide derivatives of Formula (I) for the preparation of a pharmaceutical formulation for the prevention and/or treatment of diseases and disorders associated with Mycobacterium tuberculosis Protein Tyrosine Phosphatases (MPTP), especially MPTPB.

In a third aspect, the invention provides the use of sulfonamide derivatives of Formula (I) for the preparation of a pharmaceutical formulation for the modulation, notably the inhibition of the activity or function of Mycobacterium tuberculosis Protein Tyrosine Phosphatases (MPTP), especially MPTPB.

In a fourth aspect, the invention provides a compound according to Formula (I).

In a fifth aspect, the invention provides a compound according to Formula (I) for use as a medicament.

In a sixth aspect, the invention provides a pharmaceutical composition comprising at least one a compound according to Formula (I) and a pharmaceutically acceptable carrier, diluent or excipient thereof.

In a seventh aspect, the invention provides a method for treating a patient suffering from a bacterial disorder selected from tuberculosis and other diseases and disorders associated with Mycobacterium tuberculosis Protein Tyrosine Phosphatases (MPTP), especially MPTPB. The method comprises administering a compound according to Formula (I).

In an eighth aspect, the invention provides a method of synthesis of a compound according to Formula (I).

In a ninth aspect, the invention provides compounds according to Formula (Ia).

### DETAILED DESCRIPTION OF THE INVENTION

The following paragraphs provide definitions of the various chemical moieties that make up the compounds according to the invention and are intended to apply uniformly throughout the specification and claims unless an otherwise expressly set out definition provides a broader definition.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like. By analogy, “C₁-C₁₂-alkyl” refers to monovalent alkyl groups having 1 to 12 carbon atoms, including “C₁-C₆-alkyl” groups and heptyl, octyl, nonyl, decanoyl, undecanoyl and dodecanoyl groups and “C₁-C₁₀-alkyl” refers to monovalent alkyl groups having 1 to 10 carbon atoms, “C₁-C₈-alkyl” refers to monovalent alkyl groups having 1 to 8 carbon atoms and “C₁-C₅-alkyl” refers to monovalent alkyl groups having 1 to 5 carbon atoms, “C₇-C₁₂-alkyl” refers to monovalent alkyl groups having 7 to 12 carbon atoms such as dodecyl.

“Heteroalkyl” refers to C₁-C₁₂-alkyl, preferably C₁-C₆-alkyl, wherein at least one carbon has been replaced by a heteroatom selected from O, N or S, including 2-methoxy ethyl. “Aryl” refers to an unsaturated aromatic carbocyclic group of from 6 to 14 carbon atoms having a single ring (e.g., phenyl) or multiple condensed rings (e.g., naphthyl). Aryl include phenyl, naphthyl, phenantrenyl and the like.

“C₁-C₆-alkyl aryl” refers to aryl groups having a C₁-C₆-alkyl substituent, including methyl phenyl, ethyl phenyl and the like.

“Aryl C₁-C₆-alkyl” refers to C₁-C₆-alkyl groups having an aryl substituent, including 3-phenylpropanoyl, benzyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to heteroaryl groups having a C₁-C₆-alkyl substituent, including methyl furyl and the like.

“Heteroaryl C₁-C₆-alkyl” refers to C₁-C₆-alkyl groups having a heteroaryl substituent, including furyl methyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to an aryl groups having a C₂-C₆-alkenyl substituent, including vinyl phenyl and the like.

“Aryl C₂-C₆-alkenyl” refers to a C₂-C₆-alkenyl groups having an aryl substituent, including phenyl vinyl and the like.

“C₂-C₆-alkenyl heteroaryl” refers to heteroaryl groups having a C₂-C₆-alkenyl substituent, including vinyl pyridinyl and the like.

“Heteroaryl C₂-C₆-alkenyl” refers to C₂-C₆-alkenyl groups having a Heteroaryl substituent, including pyridinyl vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₃-C₈-cycloalkyl” refers to a saturated carbocyclic group of from 3 to 8 carbon atoms having a single ring (e.g., cyclohexyl) or multiple condensed rings (e.g., norbornyl). C₃-C₈-cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Heterocycloalkyl include pyrrolidine, piperidine, piperazine, morpholine, tetrahydrofurane and the like.

“C₁-C₆-alkyl cycloalkyl” refers to C₃-C₈-cycloalkyl groups having a C₁-C₆-alkyl substituent, including methyl cyclopentyl and the like.

“Cycloalkyl C₁-C₆-alkyl” refers to C₁-C₆-alkyl groups having a C₃-C₈-cycloalkyl substituent, including 3-cyclopentyl propyl and the like.

“C₁-C₆-alkyl heterocycloalkyl” refers to heterocycloalkyl groups having a C₁-C₆-alkyl substituent, including 1-methylpiperazine and the like.

“Heterocycloalkyl C₁-C₆-alkyl” refers to C₁-C₆-alkyl groups having a heterocycloalkyl substituent, including 4-methyl piperidyl and the like.

“Carboxy” refers to the group —C(O)OH.

“Carboxy C₁-C₆-alkyl” refers to C₁-C₆-alkyl groups having an carboxy substituent, including 2-carboxyethyl and the like.

“Acyl” refers to the group —C(O)R where R includes H, “C₁-C₁₂-alkyl”, preferably “C₁-C₆-alkyl”, “aryl”, “heteroaryl”, “C₃-C₈-cycloalkyl”, “heterocycloalkyl”, “aryl C₁-C₆-alkyl”, “heteroaryl C₁-C₆-alkyl”, “C₃-C₈-cycloalkyl C₁-C₆-alkyl” or “heterocycloalkyl C₁-C₆-alkyl”.

“Acyl C₁-C₆-alkyl” to C₁-C₆-alkyl groups having an acyl substituent, including acetyl, 2-acetylethyl and the like.

“Acyl aryl” refers to aryl groups having an acyl substituent, including 2-acetylphenyl and the like.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Acyloxy C₁-C₆-alkyl” refers to C₁-C₆-alkyl groups having an acyloxy substituent, including propionic acid ethyl ester and the like.

“Alkoxy” refers to the group —O—R where R includes “C₁-C₆-alkyl” or “aryl” or “heteroaryl” or “aryl C₁-C₆-alkyl” or “heteroaryl C₁-C₆-alkyl”. Preferred alkoxy groups include for example, methoxy, ethoxy, phenoxy and the like.

“Alkoxy C₁-C₆-alkyl” refers to alkoxy groups having a C₁-C₆-alkyl substituent, including methoxy, methoxyethyl and the like.

“Alkoxycarbonyl” refers to the group —C(O)OR where R includes H, “C₁-C₆-alkyl” or “aryl” or “heteroaryl” or “aryl C₁-C₆-alkyl” or “heteroaryl C₁-C₆-alkyl” or “heteroalkyl”. “Alkoxycarbonyl C₁-C₆-alkyl” refers to C₁-C₅-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen or C₁-C₆-alkyl or aryl or heteroaryl or “aryl C₁-C₆-alkyl” or “heteroaryl C₁-C₆-alkyl”, including N-phenyl formamide.

“Aminocarbonyl C₁-C₆-alkyl” refers to C₁-C₆-alkyl groups having an aminocarbonyl substituent, including 2-(dimethylaminocarbonyl)ethyl, N-ethyl acetamide, N,N-Diethyl-acetamide and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Acylamino C₁-C₆-alkyl” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”, and where R′ and R″, together with the nitrogen atom to which they are attached, can optionally form a 3-8-membered heterocycloalkyl ring.

“Ureido C₁-C₆-alkyl” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Amino” refers to the group —NRR′ where each R,R′ is independently hydrogen or “CC₁-C₆-alkyl” or “aryl” or “heteroaryl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“Amino C₁-C₆-alkyl” refers to C₁-C₅-alkyl groups having an amino substituent, including 2-(1-pyrrolidinyl)ethyl and the like.

“Ammonium” refers to a positively charged group —N⁺RR′R″, where each R, R′, R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“Ammonium C₁-C₆-alkyl” refers to C₁-C₆-alkyl groups having an ammonium substituent, including 1-ethylpyrrolidinium and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfonyloxy C₁-C₆-alkyl” refers to C₁-C₆-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfonyl C₁-C₆-alkyl” refers to C₁-C₅-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfinyl C₁-C₆-alkyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “alkynylheteroaryl C₂-C₆”, “cycloalkyl C₁-C₆-alkyl”, “heterocycloalkyl C₁-C₆-alkyl”. Preferred sulfanyl groups include methylsulfanyl, ethylsulfanyl, and the like.

“Sulfanyl C₁-C₆-alkyl” refers to C₁-C₅-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfonylamino C₁-C₆-alkyl” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Aminosulfonyl C₁-C₆-alkyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkenyl”, “alkynyl”, “aryl”, “heteroaryl”, “cycloalkyl”, “heterocycloalkyl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like.

“Substituted” refers to groups substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “aminosulfonyl”, “ammonium”, “acyl amino”, “amino carbonyl”, “aryl”, “heteroaryl”, “sulfinyl”, “sulfonyl”, “alkoxy”, “alkoxy carbonyl”, “carbamate”, “sulfanyl”, “halogen”, trihalomethyl, cyano, hydroxy, mercapto, nitro, and the like “Pharmaceutically acceptable salts or complexes” refers to salts or complexes of the below-specified compounds of Formula (I). Examples of such salts include, but are not restricted, to base addition salts formed by reaction of compounds of formula (I) with organic or inorganic bases such as hydroxide, carbonate or bicarbonate of a metal cation such as those selected in the group consisting of alkali metals (sodium, potassium or lithium), alkaline earth metals (e.g. calcium or magnesium), or with an organic primary, secondary or tertiary alkyl amine. Amine salts derived from methylamine, dimethylamine, trimethylamine, ethylamine, diethylamine, triethylamine, morpholine, N-Me-D-glucamine, N,N′-bis(phenylmethyl)-1,2-ethanediamine, tromethamine, ethanolamine, diethanolamine, ethylenediamine, N-methylmorpholine, procaine, piperidine, piperazine and the like are contemplated being within the scope of the instant invention.

Also comprised are salts which are formed from to acid addition salts formed with inorganic acids (e.g. hydrochloric acid, hydrobromic acid, sulfuric acid, phosphoric acid, nitric acid, and the like), as well as salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid.

“Pharmaceutically active derivative” refers to any compound that upon administration to the recipient, is capable of providing directly or indirectly, the activity disclosed herein. The term “indirectly” also encompasses prodrugs which may be converted to the active form of the drug via endogenous enzymes or metabolism.

It has now been found that compounds of the present invention are modulators of the Mycobacterium tuberculosis Protein Tyrosine Phosphatases (MPTP). When the MPTP is inhibited by the compounds of the present invention, MPTP is unable to exert its biological and/or pharmacological effects.

The compounds according to Formula (I) are suitable for the modulation, notably the inhibition of the activity of Mycobacterium tuberculosis Protein Tyrosine Phosphatases. It is therefore believed that the compounds of the present invention are also particularly useful in the treatment and/or prevention of disorders which are mediated by Mycobacterium tuberculosis Protein Tyrosine Phosphatases (MPTP). Said treatment involves the modulation—notably the inhibition or the down regulation—of the Mycobacterium tuberculosis Protein Tyrosine Phosphatases (MPTP), especially MPTPB. The compounds according to the invention may be useful for the treatment and/or prevention of bacterial diseases such as tuberculosis and other diseases where immunosuppression by TB is involved. Example of such disease are pneumonia or AIDS. HIV and TB form a lethal combination, each speeding the other's progress. HIV weakens the immune system and someone who is HIV-positive and infected with TB is many times more likely to become sick with TB than someone infected with TB who is HIV-negative.

In another embodiment, the compounds of the invention can be used in the treatment of bacterial infections, especially tuberculosis and other diseases where immunosuppression by TB is involved, alone or in combination with a co-agent useful in the treatment of bacterial infections, wherein the co-agent is for example selected from the following compounds and the like:

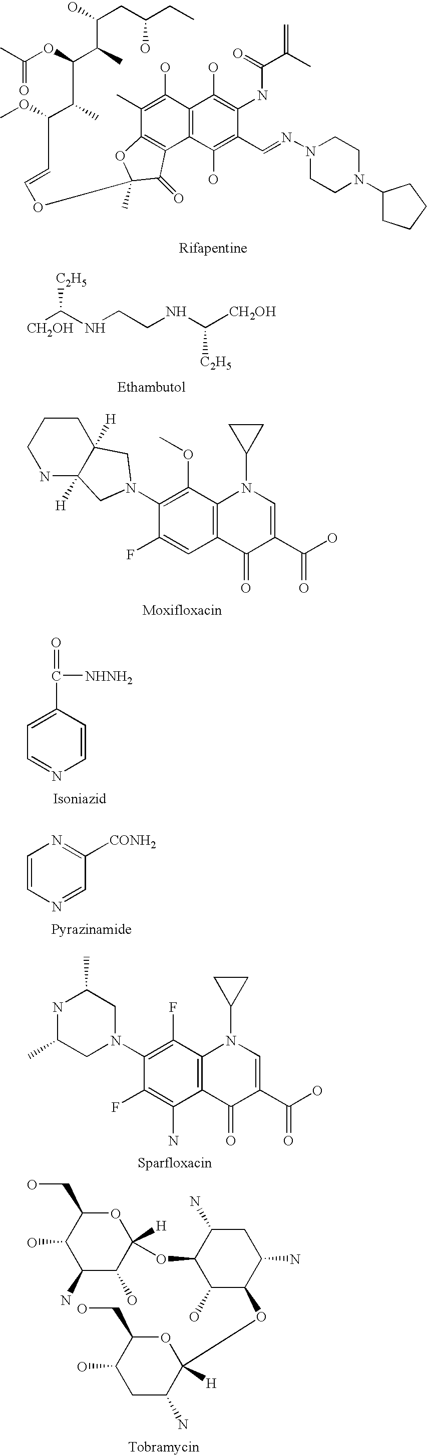

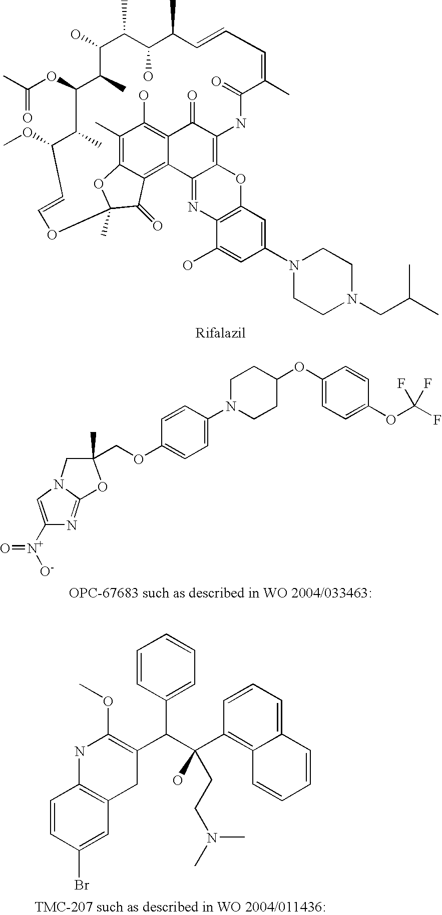

The compound of the invention and a co-agent useful in the treatment of bacterial infections can be for simultaneous, separate or sequential use in the treatment of bacterial infections.

General Formula (I) according to the present invention also comprises its tautomers, its geometrical isomers, its optically active forms as enantiomers, diastereomers and its racemate forms, as well as pharmaceutically acceptable salts thereof. Preferred pharmaceutically acceptable salts of the Formula (I) are amine salts derived from methylamine, dimethylamine, trimethylamine, ethylamine, diethylamine, triethylamine, morpholine, N-Me-D-glucamine, N,N′-bis(phenylmethyl)-1,2-ethanediamine, tromethamine, ethanolamine, diethanolamine, ethylenediamine, N-methylmorpholine, procaine, piperidine, piperazine and the like salts.

In one embodiment, the invention provides the use of sulfonamide derivatives of Formula (I):

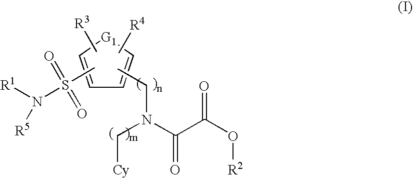

Wherein G¹ is selected from —CR⁶R⁷—, —O—, —S— and —N(R⁸);

R¹ is selected from H; optionally substituted C₁-C₆ alkyl, optionally substituted C₇-C₁₂-alkyl including dodecyl, hexyl, 2-ethyl hexyl, optionally substituted acyloxy C₁-C₆-alkyl such as butyric acid; optionally substituted C₂-C₆ alkenyl; optionally substituted C₂-C₆ alkynyl; optionally substituted aryl C₁-C₆ alkyl, including optionally substituted phenyl C₁-C₆ alkyl (e.g. 3,3-diphenylpropyl, 2-(4-phenoxy phenyl)ethyl, 2-biphenyl-4-ylethyl, 2-(4-chlorophenyl)propyl); optionally substituted heteroaryl C₁-C₆ alkyl; optionally substituted C₃-C₈ cycloalkyl C₁-C₆ alkyl and optionally substituted heteroaryl C₁-C₆ alkyl;

R² is selected from H, optionally substituted C₁-C₆ alkyl, C₂-C₆ alkenyl; optionally substituted C₂-C₆ alkynyl and optionally substituted C₃-C₈ cycloalkyl;

R³ and R⁴ are independently selected from H, optionally substituted C₁-C₆ alkyl and halogen;

R⁵ is selected from H, optionally substituted C₁-C₆ alkyl, C₂-C₆ alkenyl; optionally substituted C₂-C₆ alkynyl and optionally substituted C₃-C₈ cycloalkyl;

R⁶, R⁷ and R⁸ are independently selected from H, optionally substituted C₁-C₆ alkyl, C₂-C₆ alkenyl and optionally substituted C₂-C₆ alkynyl;

Cy is selected from optionally substituted aryl, including optionally substituted phenyl such as phenyl, halogeno phenyl (e.g. m-chloro phenyl, o-fluoro phenyl), trifluoromethyl phenyl (e.g. m-trifluoromethyl phenyl, p-trifluoromethyl phenyl), nitro phenyl (e.g. m-nitro phenyl), phenoxy phenyl (e.g. p-phenoxy phenyl), methoxy phenyl (e.g. p-methoxy phenyl), benzoic acid (e.g. p-benzoic acid); optionally substituted heteroaryl, including optionally substituted pyridine such as pyridine (e.g. 4-pyridinyl), trifluoromethyl pyridinyl (e.g. p-trifluoromethyl pyridinyl); optionally substituted C₃-C₈ cycloalkyl, including optionally substituted cyclopentyl and optionally substituted heterocycloalkyl;

m and n are integers independently selected from 0, 1, 2, 3 and 4; as well as its geometrical isomers, its optically active forms as enantiomers, diastereomers and its racemate forms, as well as pharmaceutically acceptable salts thereof for the preparation of a pharmaceutical formulation for the treatment of bacterial infections such as tuberculosis.

In a specific embodiment, the invention provides sulfonamide derivatives of Formula (I) wherein G¹ is —S—.

In another specific embodiment, the invention provides sulfonamide derivatives of Formula (I) wherein R¹ is selected from optionally substituted C₁-C₆ alkyl, optionally substituted C₇-C₁₂-alkyl optionally substituted C₂-C₆ alkenyl and optionally substituted C₂-C₆ alkynyl.

In another specific embodiment, the invention provides sulfonamide derivatives of Formula (I) wherein R¹ is selected from optionally substituted aryl C₁-C₆ alkyl and optionally substituted heteroaryl C₁-C₆ alkyl.

In another specific embodiment, the invention provides sulfonamide derivatives of Formula (I) wherein R² is H.

In another specific embodiment, the invention provides sulfonamide derivatives of Formula (I) wherein R³ is H.

In another specific embodiment, the invention provides sulfonamide derivatives of Formula (I) wherein R⁴ is H.

In another specific embodiment, the invention provides sulfonamide derivatives of Formula (I) wherein R⁵ is H.

In another specific embodiment, the invention provides sulfonamide derivatives of Formula (I) wherein R⁶ is H.

In another specific embodiment, the invention provides sulfonamide derivatives of Formula (I) wherein R⁷ is H.

In another specific embodiment, the invention provides sulfonamide derivatives of Formula (I) wherein R⁸ is H.

In another specific embodiment, the invention provides sulfonamide derivatives of Formula (I) wherein m is 0.

In another specific embodiment, the invention provides sulfonamide derivatives of Formula (I) wherein m is 1.

In another specific embodiment, the invention provides sulfonamide derivatives of Formula (I) wherein n is 0.

In another specific embodiment, the invention provides sulfonamide derivatives of Formula (I) wherein n is 1.

In another specific embodiment, the invention provides sulfonamide derivatives of Formula (I) wherein R², R³, R⁴, R⁵, R⁶, R⁷ and R⁸ are H; M is selected from 0 and 1; n is 1; G¹ is —S—; R¹ and Cy are as described in the description.

Compounds of the present invention include in particular those of the group consisting of:

| Example | Name |
|---|---|
| 1 | {({5-[(dodecylamino)sulfonyl]-2-thienyl}methyl)[4-(trifluoromethyl)benzyl] |
| | amino}(oxo)acetic acid; |
| 2 | [benzyl({5-[(dodecylamino)sulfonyl]-2-thienyl}methyl)amino](oxo)acetic acid; |
| 3 | [cyclopentyl({5-[(dodecylamino)sulfonyl]-2-thienyl}methyl)amino](oxo)acetic |
| | acid; |
| 4 | [({5-[(dodecylamino)sulfonyl]-2-thienyl}methyl)(3-nitrobenzyl)amino](oxo)acetic |
| | acid; |
| 5 | [({5-[(dodecylamino)sulfonyl]-2-thienyl}methyl)(4-methoxybenzyl)amino] |
| | (oxo)acetic acid; |
| 6 | [({5-[(dodecylamino)sulfonyl]-2-thienyl}methyl)(2-fluorobenzyl)amino](oxo)acetic |
| | acid; |
| 7 | {({5-[(dodecylamino)sulfonyl]-2-thienyl}methyl)[4-(methylsulfonyl)benzyl] |
| | amino}(oxo)acetic acid; |
| 8 | [({5-[(dodecylamino)sulfonyl]-2-thienyl}methyl)(4-phenoxybenzyl)amino] |
| | (oxo)acetic acid; |
| 9 | 4-{[(carboxycarbonyl)({5-[(dodecylamino)sulfonyl]-2-thienyl}methyl)amino] |
| | methyl}benzoic acid; |
| 10 | (({5-[(dodecylamino)sulfonyl]-2-thienyl}methyl){[6-(trifluoromethyl)pyridin-3- |
| | yl]methyl}amino)(oxo)acetic acid; |
| 11 | {({5-[(dodecylamino)sulfonyl]-2-thienyl}methyl)[3-(trifluoromethyl)benzyl] |
| | amino}(oxo)acetic acid; |
| 12 | [(3-chlorobenzyl)({5-[(dodecylamino)sulfonyl]-2-thienyl}methyl)amino](oxo)acetic |
| | acid; |
| 13 | {[(5-{[(3,3-diphenylpropyl)amino]sulfonyl}-2-thienyl)methyl][3-(trifluoromethyl) |
| | benzyl]amino}(oxo)acetic acid; |
| 14 | {(3-chlorobenzyl)[(5-{[(3,3-diphenylpropyl)amino]sulfonyl)-2-thienyl)methyl] |
| | amino}(oxo)acetic acid; |
| 15 | oxo{{[5-({[2-(4-phenoxyphenyl)ethyl]amino}sulfonyl)-2-thienyl]methyl}[3- |
| | (trifluoromethyl)benzyl]amino)acetic acid; |
| 16 | ((3-chlorobenzyl){[5-({[2-(4-phenoxyphenyl)ethyl]amino}sulfonyl)-2- |
| | thienyl]methyl)amino)(oxo)acetic acid; |
| 17 | {[(5-{[(2-biphenyl-4-ylethyl)amino]sulfonyl}-2-thienyl)methyl][3-(trifluoromethyl) |
| | benzyl]amino}(oxo)acetic acid; |
| 18 | {({5-[(hexylamino)sulfonyl]-2-thienyl}methyl)[3-(trifluoromethyl)benzyl] |
| | amino}(oxo)acetic acid; |
| 19 | [[(5-{[(2-biphenyl-4-ylethyl)amino]sulfonyl}-2-thienyl)methyl](pyridin-4-ylmethyl) |
| | amino](oxo)acetic acid; |
| 20 | 4-({[5-({(carboxycarbonyl)[3-(trifluoromethyl)benzyl]amino}methyl)-2-thienyl] |
| | sulfonyl}amino)butanoic acid; |
| 21 | 4-{[(5-{[(carboxycarbonyl)(3-chlorobenzyl)amino]methyl}-2-thienyl)sulfonyl] |
| | amino}butanoic acid; |
| 22 | {{[5-({[2-(4-chlorophenyl)propyl]amino}sulfonyl)-2-thienyl]methyl}[3-(trifluoro |
| | methyl)benzyl]amino}(oxo)acetic acid; |
| 23 | [{[5-({[2-(4-chlorophenyl)propyl]amino}sulfonyl)-2-thienyl]methyl}(pyridin-4- |
| | ylmethyl)amino](oxo)acetic acid; |
| 24 | ((3-chlorobenzyl){[5-({[2-(4-chlorophenyl)propyl]amino}sulfonyl)-2-thienyl] |
| | methyl}amino)(oxo)acetic acid; |
| 25 | {[(5-{[(2-ethylhexyl)amino]sulfonyl}-2-thienyl)methyl][3-(trifluoromethyl) |
| | benzyl]amino}(oxo)acetic acid; |
| 26 | [[(5-{[(2-ethylhexyl)amino]sulfonyl}-2-thienyl)methyl](pyridin-4-ylmethyl) |
| | amino](oxo)acetic acid; |
| 27 | {(3-chlorobenzyl)[(5-{[(2-ethylhexyl)amino]sulfonyl}-2-thienyl)methyl] |
| | amino}(oxo)acetic acid; |
| 28 | ((3-chlorobenzyl){[5-({[2-({[5-(dimethylamino)-1-naphthyl]sulfonyl}amino) |
| | ethyl]amino}sulfonyl)-2-thienyl]methyl}amino)(oxo)acetic acid. |

In another specific embodiment, the invention provides a use of compounds of Formula (I) for the preparation of a pharmaceutical formulation for the prevention and/or treatment of diseases and disorders associated with Mycobacterium tuberculosis Protein Tyrosine Phosphatases (MPTP), especially MPTPB.

In another specific embodiment, the invention provides a use of compounds of Formula (I) for the preparation of a pharmaceutical formulation for the modulation, notably the inhibition of the activity or function of Mycobacterium tuberculosis Protein Tyrosine Phosphatases (MPTP), especially MPTPB.

In another specific embodiment, the invention provides a compound according to Formula (I) selected from the list below:
[[(5-{[(2-biphenyl-4-ylethyl)amino]sulfonyl}-2-thienyl)methyl](pyridin-4-ylmethyl)amino](oxo)acetic acid;
4-({[5-({(carboxycarbonyl)[3-(trifluoromethyl)benzyl]amino}methyl)-2-thienyl]sulfonyl}amino)butanoic acid;
4-{[(5-{[(carboxycarbonyl)(3-chlorobenzyl)amino]methyl}-2-thienyl)sulfonyl]amino}butanoic acid;
{{[5-({[2-(4-chlorophenyl)propyl]amino}sulfonyl)-2-thienyl]methyl}[3-(trifluoromethyl)benzyl]amino}(oxo)acetic acid;
[{[5-({[2-(4-chlorophenyl)propyl]amino}sulfonyl)-2-thienyl]methyl}(pyridin-4-ylmethyl)amino](oxo)acetic acid;
((3-chlorobenzyl) {[5-({[2-(4-chlorophenyl)propyl]amino}sulfonyl)-2-thienyl]methyl}amino)(oxo)acetic acid;
{[(5-{[(2-ethylhexyl)amino]sulfonyl}-2-thienyl)methyl][3-(trifluoromethyl)benzyl]amino}(oxo)acetic acid;
[[(5-{[(2-ethylhexyl)amino]sulfonyl}-2-thienyl)methyl](pyridin-4-ylmethyl)amino](oxo) acetic acid;
{(3-chlorobenzyl) [(5-{[(2-ethylhexyl)amino]sulfonyl}-2-thienyl)methyl]amino}(oxo) acetic acid;
((3-chlorobenzyl) {[5-({[2-({[5-(dimethylamino)-1-naphthyl]sulfonyl}amino)ethyl]amino}sulfonyl)-2-thienyl]methyl}amino)(oxo)acetic acid.

In another specific embodiment, the invention provides a compound according to Formula (I) for use as a medicament selected from the list below:
[[(5-{[(2-biphenyl-4-ylethyl)amino]sulfonyl}-2-thienyl)methyl](pyridin-4-ylmethyl)amino](oxo)acetic acid;
4-({[5-({(carboxycarbonyl)[3-(trifluoromethyl)benzyl]amino}methyl)-2-thienyl]sulfonyl}amino)butanoic acid;
4-{[(5-{[(carboxycarbonyl)(3-chlorobenzyl)amino]methyl}-2-thienyl)sulfonyl]amino}butanoic acid;
{{[5-({[2-(4-chlorophenyl)propyl]amino}sulfonyl)-2-thienyl]methyl}[3-(trifluoromethyl)benzyl]amino}(oxo)acetic acid;
[{[5-({[2-(4-chlorophenyl)propyl]amino}sulfonyl)-2-thienyl]methyl}(pyridin-4-ylmethyl)amino](oxo)acetic acid;
((3-chlorobenzyl) {[5-({[2-(4-chlorophenyl)propyl]amino}sulfonyl)-2-thienyl]methyl}amino)(oxo)acetic acid;
{[(5-{[(2-ethylhexyl)amino]sulfonyl}-2-thienyl)methyl][3-(trifluoromethyl)benzyl]amino}(oxo)acetic acid;
[[(5-{[(2-ethylhexyl)amino]sulfonyl}-2-thienyl)methyl](pyridin-4-ylmethyl)amino](oxo) acetic acid;
{(3-chlorobenzyl) [(5-{[(2-ethylhexyl)amino]sulfonyl}-2-thienyl)methyl]amino}(oxo) acetic acid;
((3-chlorobenzyl) {[5-({[2-({[5-(dimethylamino)-1-naphthyl]sulfonyl}amino)ethyl]amino}sulfonyl)-2-thienyl]methyl}amino)(oxo)acetic acid.

In another specific embodiment, the invention provides a pharmaceutical composition comprising at least one a compound according to Formula (I) and a pharmaceutically acceptable carrier, diluent or excipient thereof selected from the list below:
[[(5-{[(2-biphenyl-4-ylethyl)amino]sulfonyl}-2-thienyl)methyl](pyridin-4-ylmethyl)amino](oxo)acetic acid;
4-({[5-({(carboxycarbonyl)[3-(trifluoromethyl)benzyl]amino}methyl)-2-thienyl]sulfonyl}amino)butanoic acid;
4-{[(5-{[(carboxycarbonyl)(3-chlorobenzyl)amino]methyl}-2-thienyl)sulfonyl]amino}butanoic acid;
{{[5-({[2-(4-chlorophenyl)propyl]amino}sulfonyl)-2-thienyl]methyl}[3-(trifluoromethyl)benzyl]amino}(oxo)acetic acid;
[{[5-({[2-(4-chlorophenyl)propyl]amino}sulfonyl)-2-thienyl]methyl}(pyridin-4-ylmethyl)amino](oxo)acetic acid;
((3-chlorobenzyl) {[5-({[2-(4-chlorophenyl)propyl]amino}sulfonyl)-2-thienyl]methyl}amino) (oxo)acetic acid;
{[(5-{[(2-ethylhexyl)amino]sulfonyl}-2-thienyl)methyl][3-(trifluoromethyl)benzyl]amino}(oxo)acetic acid;
[[(5-{[(2-ethylhexyl)amino]sulfonyl}-2-thienyl)methyl](pyridin-4-ylmethyl)amino](oxo) acetic acid;
{(3-chlorobenzyl) [(5-{[(2-ethylhexyl)amino]sulfonyl}-2-thienyl)methyl]amino}(oxo) acetic acid;
((3-chlorobenzyl) {[5-({[2-({[5-(dimethylamino)-1-naphthyl]sulfonyl}amino)ethyl]amino}sulfonyl)-2-thienyl]methyl}amino)(oxo)acetic acid.

In another specific embodiment, the invention provides a method for treating a patient suffering from a bacterial disorder selected from tuberculosis and other diseases and disorders associated with Mycobacterium tuberculosis Protein Tyrosine Phosphatases (MPTP), especially MPTPB. The method comprises administering a compound according to Formula (I).

In another embodiment according to the invention, is provided a process for the preparation of sulfonamide derivative according to Formula (I), comprising the step of reacting an amine of Formula (II) with a carboxylic acid of Formula LG₂-CO—CO—R²

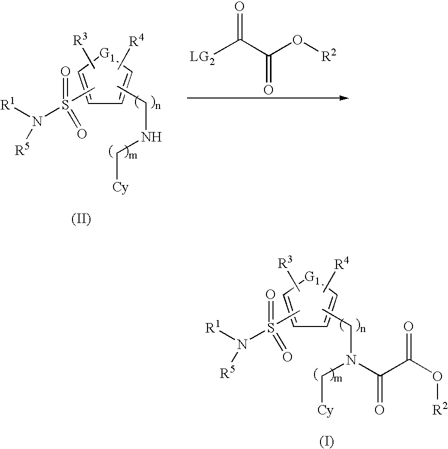

Wherein R¹, R², R³, R⁴, R⁵, R⁶, R⁷, m, n is 1, G¹ and Cy are as described in the detailed description and LG₂ is a suitable leaving group—including Cl, N-hydroxy succinimide or benzotriazol-1-yl.

In another embodiment according to the invention, is provided a derivative of Formula (II) selected from the following group:
Ethyl {({5-[(hexylamino)sulfonyl]-2-thienyl}methyl)[3 (trifluoromethyl)benzyl]amino}(oxo)acetate;
Ethyl [[(5-{[(2-biphenyl-4-ylethyl)amino]sulfonyl}-2-thienyl)methyl](pyridin-4-ylmethyl)amino](oxo)acetate;
4-({[5-({[ethoxy(oxo)acetyl][3-(trifluoromethyl)benzyl]amino}methyl)-2-thienyl]sulfonyl}amino)butanoic acid;
4-({[5-({(3-chlorobenzyl)[ethoxy(oxo)acetyl]amino}methyl)-2-thienyl]sulfonyl}amino)butanoic acid;
Ethyl {{[5-({[2-(4-chlorophenyl)propyl]amino}sulfonyl)-2-thienyl]methyl}[3-(trifluoro methyl)benzyl]amino}(oxo)acetate;
Ethyl [{[5-({[2-(4-chlorophenyl)propyl]amino}sulfonyl)-2-thienyl]methyl}(pyridin-4-yl methyl)amino](oxo)acetate;
Ethyl ((3-chlorobenzyl) {[5-({[2-(4-chlorophenyl)propyl]amino}sulfonyl)-2-thienyl]methyl}amino)(oxo)acetate;
Ethyl {[(5-{[(2-ethylhexyl)amino]sulfonyl}-2-thienyl)methyl][3-(trifluoromethyl)benzyl]amino}(oxo)acetate;
Ethyl [[(5-{[(2-ethylhexyl)amino]sulfonyl}-2-thienyl)methyl](pyridin-4-ylmethyl)amino](oxo)acetate;
Ethyl {(3-chlorobenzyl)[(5-{[(2-ethylhexyl)amino]sulfonyl}-2-thienyl)methyl]amino}(oxo)acetate;
Ethyl ((3-chlorobenzyl) {[5-({[2-({[5-(dimethylamino)-1-naphthyl]sulfonyl}amino)ethyl]amino}sulfonyl)-2-thienyl]methyl}amino)(oxo)acetate.

The sulfonamide derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

When employed as pharmaceuticals, the compounds of the present invention are typically administered in the form of a pharmaceutical composition. Hence, pharmaceutical compositions comprising a compound of Formula (I) and a pharmaceutically acceptable carrier, diluent or excipient therefore are also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Pharmaceutical compositions containing sulfonamide derivatives of this invention can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the sulfonamide derivative is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As above mentioned, the sulfonamide derivatives of Formula (I) in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

### Synthesis of Compounds of the Invention:

The sulfonamide derivatives according to Formula (I) can be prepared from readily available starting materials by several synthetic approaches, using both solution-phase and solid-phase chemistry protocols. Examples of synthetic pathways for the will be described.

The following abbreviations refer respectively to the definitions below:

min (minute), h (hour), g (gram), mg (milligram), mmol (millimole), m.p. (melting point), eq (equivalents), mL (milliliter), μL (microliters), mL (milliliters), APCI (Atmospheric pressure chemical ionization), ESI (Electro-spray ionization), L (liters), AcOEt (Ethyl acetate), Boc (tert-Butoxycarbonyl), CH₃CN (Acetonitrile), DCC (Dicyclohexyl carbodiimide), DCE (Dichloroethane), DIEA (Diisopropylethylamine), Fmoc (9-Fluorenylmethoxycarbonyl), CDCl₃ (deuterated chloroform), c-Hex (Cyclohexanes), DCM (Dichloromethane), DIC (Diisopropyl carbodiimide), DMAP (4-Dimethylaminopyridine), DMF (Dimethylformamide), DMSO (Dimethylsulfoxide), DMSO-d₆ (Deuterated dimethylsul-foxide), EDC (1-(3-Dimethyl-amino-propyl)-3-ethylcarbodiimide), EtOAc (Ethyl acetate), Et₂O (Diethyl ether), EtOH (Ethanol), HOBt (1-Hydroxybenzotriazole), K₂CO₃ (Potassium carbonate), MeOH (Methanol), CD₃OD (Deuterated methanol), MgSO₄ (Magnesium sulfate), NaH (Sodium hydride), NaHCO₃ (Sodium bicarbonate), NaBH₃CN (Sodium cyanoborohydride), NaBH₄ (Sodium borohydride), NaBH(OAc)₃ (Sodium triacetoxyborohydride), NMM (N-methyl-morpholine), NMP (N-Methylpyrrolidone), PetEther (Petroleum ether), Pht (Phtalimide), PyBOP® (Bentotriazole-1-yl-oxy-tris-pyrrolidino-phosphonium hexafluorophosphate), rt (room temperature), Rt (retention time), SPE (solid phase extraction), TEA (Triethylamine), TFA (Trifluoroacetic acid), THF (Tetrahydrofuran), HAc (Acetic acid), TMOF (Trimethyorthoformiate).

The sulfonamide derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

Examples of methods of synthesis of sulfonamides according to the invention are given in WO 03/064376 and in the process illustrated in the following schemes.

### Methods of Preparing Intermediates of Compounds of Formula (I).

Generally, derivatives according to the general formula (I) may be obtained by several processes, using both solution-phase and solid-phase chemistry protocols. Depending on the nature of G₁, R¹, R³, R⁴, R⁵, R⁶, R⁷, R⁸, Cy m and n, some processes will be preferred to others, this choice of the most suitable process being assumed by the practitioner skilled in the art.

### a) Preparation Using Solution Phase:

Generally, derivatives of formula (I) may be obtained by the initial synthesis of the esters (Ia) and their subsequent hydrolysis to give rise to the derivatives of the general formula (I).

In the following the general preparation of derivatives of Formula (J), wherein G₁, R¹, R³, R⁴, R⁵, R⁶, R⁷, R⁸, Cy m and n are as above-defined, shall be illustrated (see Scheme 1 below).

Derivatives of Formula (J) may be prepared by coupling corresponding carboxylic acid derivatives of Formula (LG₂-CO—CO—R²), wherein LG₂ is a suitable leaving group—including Cl, N-hydroxy succinimide or benzotriazol-1-yl- and wherein R² is selected from optionally substituted C₁-C₆ alkyl, C₂-C₆ alkenyl, optionally substituted C₂-C₆ alkynyl and optionally substituted C₃-C₈ cycloalkyl, and a primary or secondary amine of Formula (II).

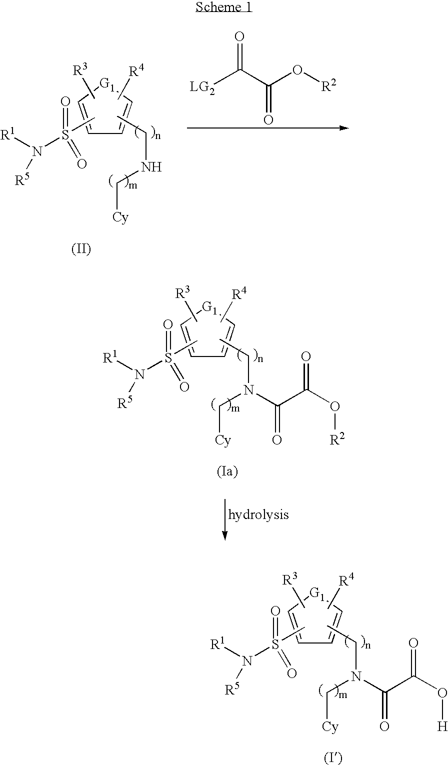

Preparation of said amide derivatives is performed using conditions and methods well known to those skilled in the art to prepare an amide bond from an amine and a carboxylic acid or carboxylic acid derivative (e.g. acid chloride), with standard coupling agents, such as e.g. DIC, EDC, TBTU, DECP, DCC, PyBOP®, Isobutyl chloroformate or others in the presence or not of bases such as TEA, DIEA, NMM in a suitable solvent such as DCM, THF or DMF. Derivatives of Formula (Ia), i.e. of Formula (J) wherein R² is selected from optionally substituted C₁-C₆ alkyl, C₂-C₆ alkenyl, optionally substituted C₂-C₆ alkynyl and optionally substituted C₃-C₈ cycloalkyl, are then submitted to hydrolysis using hydroxide (e.g. NaOH) and leading to the desired compounds of Formula (I′), i.e. of Formula (J) wherein R² is H.

Amines of Formula (II) may for instance be prepared by alkylation of the amines of Formula (III)—wherein G₁, R¹, R³, R⁴, R⁵ and n are as above-defined (Scheme 2 below). The reaction may be performed by the reductive alkylation of a compound of Formula (III) with an carbonyl reagent of Formula (VIII), wherein R^{1a} and R^{1b} are H, optionally substituted C₁-C₆ alkyl, optionally substituted aryl or can form an optionally substituted C₃-C₈ cycloalkyl, in the presence of a suitable reducing agent including NaBH(OAc)₃, NaBH₃CN, NaBH₄ or hydrogen and an appropriate catalyst such as Pd/C or PtO₂. Alternatively, amines of Formula (II) can be obtained by alkylation of amines (III) by an alkylating agent of Formula (VII) where LG1 is a leaving group such as Cl, Br, I, OTf, Ots or OMs.

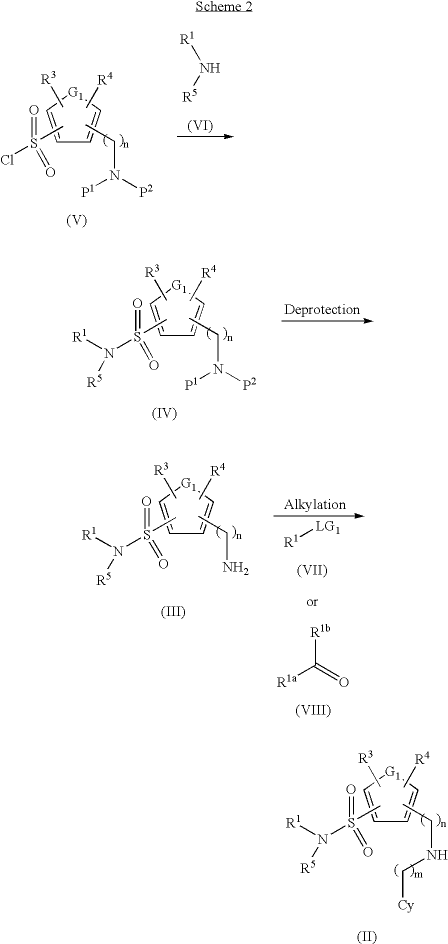

Said amines of Formula (III) may be obtained by deprotection of their corresponding protected form of Formula (IV), wherein P¹ and P2 are H or a protecting group such as e.g. Boc or Fmoc or wherein both P¹ and P² form a phtalimide protecting group. For all the protection, deprotection methods, see Kocienski, in “Protecting Groups”, Georg Thieme Verlag Stuttgart, New York, 1994 and Theodora W Greene and Peter G. M. Wuts in “Protective Groups in Organic Synthesis”, 3^{rd} edition, John Wiley & Sons Inc., 1999 (NY).

Protected amines of Formula (IV) wherein G₁, R¹, R³, R⁴ and n are defined as above can be prepared by reacting the sulfonyl chloride of Formula (V) with an amine of Formula (VI) under conditions well known for those skilled in the art.

The precursor compounds of Formulae (VI), (VII), (VIII) are either commercially available or readily accessible from commercial starting materials.

b) Preparation Using Solid-Phase and/or Mixed Solid/Solution Phase:

According to yet another general approach, derivatives according to the general Formula (I), wherein the substituents G₁, R¹, R³, R⁴, R⁶, R⁷, R⁸, Cy, m and n are as above defined, and wherein R⁵ is H, may be prepared by solid-phase and/or mixed solid/solution-phase synthesis protocols such as those described in the examples and shown in Schemes 1, 2 and 3 using well known technical approaches (such as IRORI®). It will be appreciated by the practitioner skilled in the art that basically the same conditions, methods and reagents as above described in Schemes 1, 2 and 3 for the solution-phase synthesis of compounds of Formula (I) could be applied to the solid-phase and/or mixed solid-/solution-phase synthesis of said compounds.

The filled circles in the below Scheme 3 illustrate the resin beads to which the compounds are linked during the solid phase synthesis. Cleavage from the resin is performed under acidic conditions, affording the corresponding derivatives of Formula (I). It is to be understood that further to the resin types mentioned in the Examples such as e.g. Sasrin aldehyde resins, other suitable reagents, notably resins, known to a person skilled in the art, could be employed for the solid-phase synthesis of compounds of general Formula (I).

In one particularly preferred process, resin-bound amines of Formula (VI), wherein R¹ is above-defined in the description, are prepared from commercially available per se or readily accessible from resins such as e.g. Sasrin aldehyde or bromo-Wang resins and amines, using standard reductive amination or alkylation conditions well known to the practitioner skilled in the art. The resin-bound amines (SPS-VI) may then be sulfonylated with compounds of Formula (V), where P1 and P2 are H or a protecting group such as Boc, Fmoc, or P1 and P2 form a phtalimide protecting group, in the presence of base such as e.g. DIEA, in suitable solvent such as NMP, THF or DCM affording compounds of formula (SPS-IV) (Scheme 3 below).

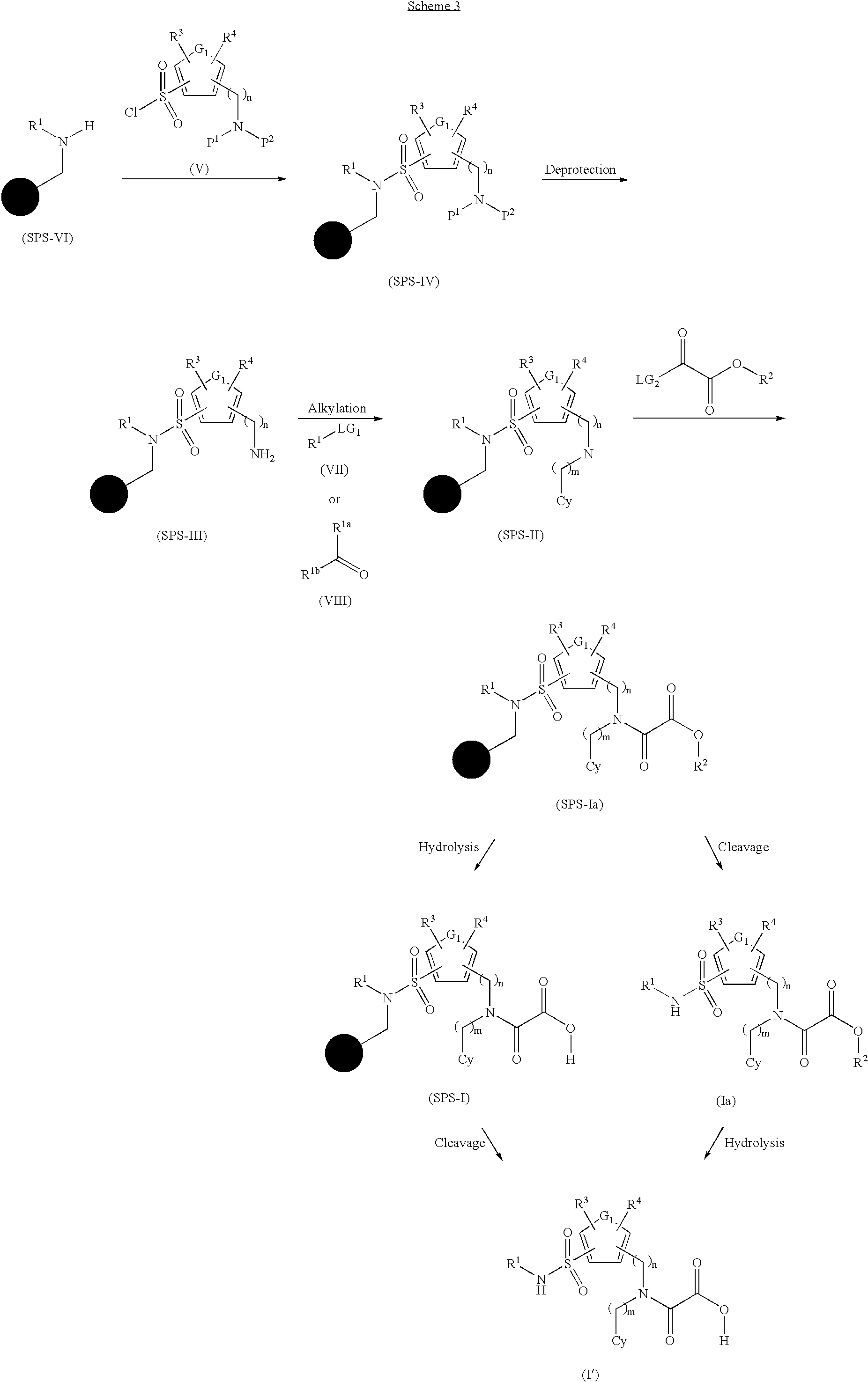

After the deprotection of the resin bound amines of Formula (SPS-IV), the alkylation may be performed by reductive alkylation of the resin bound of Formula (SPS-III) with an carbonyl reagent of Formula (VIII), wherein R^{1a} and R^{1b} are H, optionally substituted C₁-C₆ alkyl, optionally substituted aryl or can form an optionally substituted C₃-C₈ cycloalkyl, in the presence of a suitable reducing agent including NaBH(OAc)₃, NaBH₃CN, NaBH₄ or hydrogen and an appropriate catalyst. Alternatively, amines of Formula (SPS-II) can be obtained by alkylation of amines (SPS-III) by an alkylating agent of Formula (VII) where LG1 is a leaving group such as Cl, Br, I, OTf, OTs, or OMs.

Finally, those compounds of Formula (SPS-II) are coupled with the ester LG₂-CO—COO—R², wherein R² is selected from H, optionally substituted C₁-C₆ alkyl, C₂-C₆ alkenyl, optionally substituted C₂-C₆ alkynyl and optionally substituted C₃-C₈ cycloalkyl and LG₂ is a leaving group such as e.g. Cl, in the presence of a base such as DIEA in an aprotic solvent (such as e.g. DCM or THF) affording the resin-bound ester of Formula (SPS-Ia). The latter compounds can be hydrolysed to the resin bound compounds of Formula (SPS-I), wherein R² is H, by their treatment with hydroxide such as e.g. NaOH in an appropriate solvent (such as e.g. THF). Cleavage from the resin is then performed under acidic conditions (such as e.g. a DCM solution containing 20% TFA), affording the corresponding desired derivatives of Formula (I′). Alternatively, the resin-bound ester of Formula (SPS-Ia) wherein can be first cleaved to the ester of Formula (Ia) wherein R⁴ is selected from H, C₁-C₆ alkyl and halogen, then hydrolysed to the desired derivatives of Formula (I′).

According to a further general process, compounds of Formula (I) can be converted to alternative compounds of Formula (I), employing suitable interconversion techniques well known by a person skilled in the art.

If the above set of general synthetic methods is not applicable to obtain compounds according to Formula (I) and/or necessary intermediates for the synthesis of compounds of Formula (I), suitable methods of preparation known by a person skilled in the art should be used. In general, the synthesis pathways for any individual compound of Formula (I) will depend on the specific substitutents of each molecule and upon the ready availability of intermediates necessary; again such factors being appreciated by those of ordinary skill in the art. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Compounds of this invention can be isolated in association with solvent molecules by crystallization from evaporation of an appropriate solvent. The pharmaceutically acceptable base addition salts of the compounds of Formula (I), which contain an acidic center, may be prepared in a conventional manner. For example, a solution of the free acid may be treated with a suitable base, either neat or in a suitable solution, and the resulting salt isolated either by filtration or by evaporation under vacuum of the reaction solvent. Pharmaceutically acceptable acid addition salts may be obtained in an analogous manner by treating a solution of compound of Formula (I) with a suitable acid. Both types of salts may be formed or interconverted using ion-exchange resin techniques.

In the following the present invention shall be illustrated by means of some examples, which are not construed to be viewed as limiting the scope of the invention.

### EXAMPLES

### The Following Starting Materials Commercially Available were Used

Dodecylamine (from Fluka), 3,3-diphenyl-propylamine (from Aldrich), 2-(4-phenoxy-phenyl)-ethylamine (from Transworld), 2-biphenyl-4-yl-ethylamine (from Transworld), hexylamine (from Aldrich), 4-amino-butyric acid t-butyl ester, hydrochloride salt (from Sennchem), 2-(4-chloro-phenyl)-propylamine, hydrochloride salt (from Sigma), 2-ethyl-hexylamine (from Fluka), Dansyl ethylenediamine (from Molecular Probes), 4-trifluoromethyl-benzaldehyde (from Aldrich), benzaldehyde (from Aldrich), cyclopentanone (from Fluka), 3-nitro-benzaldehyde (from Fluka), 4-methoxy-benzaldehyde (from Fluka), 2-fluoro-benzaldehyde (from Fluka), 4-methanesulfonyl-benzaldehyde (from Acros), 4-phenoxy-benzaldehyde (from Aldrich), 4-formyl-benzoic acid methyl ester (from Fluka), 6-trifluoromethyl-pyridine-3-carbaldehyde (from Peakdale), 3-trifluoromethyl-benzaldehyde (from Aldrich), 3-chloro-benzaldehyde (from Fluka), thiophene-2-methylamine (from Aldrich), pyridine-4-carbaldehyde (from Fluka).

The HPLC, MS and NMR data provided in the examples described below were obtained as followed. HPLC: Waters Symmetry C₈ column 50 mm×4.6 mm; UW detection at 254 nm; flow: 2 mL/min; Conditions A: 8 min gradient from 0.1% TFA in H₂O to 0.07% TFA in CH₃CN; Conditions B: 10 min gradient from 0.1% TFA in H₂O to 0.07% TFA in CH₃CN. The semi-preparative reverse-phase HPLC was obtained as followed: Supelcosil ABZ+Plus column (25 cm×21.2 mm, 12 μm); UW detection at 254 nm and 220 nm; flow 20 mL/min; Condition C: 10 min gradient from 30% CH₃CN in 0.1% TFA in CH₃CN to 100% CH₃CN followed by 5 min elution at 100% CH₃CN. The MS data provided in the examples described below were obtained as followed: Mass spectrum: PE sciex API 150 EX (APCI or ESI) or LC/MS Waters ZMD (ESI). The NMR data provided in the examples described below were obtained as followed: ¹H-NMR: Bruker DPX-300 MHz. TLC Analysis is performed on Merck Precoated 60 F₂₅₄ plates. Purifications by flash chromatography are performed on SiO₂ support, using cyclohexane/EtOAc or DCM/MeOH mixtures as eluents.

### Example 1

### {({5-[(dodecylamino)sulfonyl]-2-thienyl}methyl) [4-(trifluoromethyl)-benzyl]amino}(oxo)acetic acid (1)

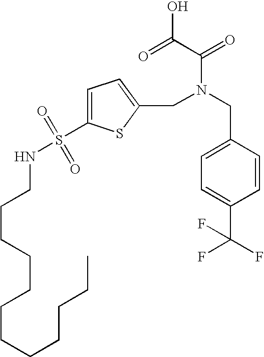

### Step a) Formation of 2-(thien-2-ylmethyl)-1H-isoindole-1,3(2H)-dione

A solution of thiophene-2-methylamine (4.2 g, 37.1 mmol) and of phtalic anhydride (5 g, 33.7 mmol) in toluene (100 mL) was stirred and heated at reflux for 3 h to remove the formed water by azeotropic distillation (Dean-Stark). The solvent was then evaporated under vacuum. The residue was dissolved in DCM (100 mL), washed with water (3×30 mL), dried over MgSO₄, filtered and concentrated to afford the title compound as a white solid (7.8 g, 95%). ¹H NMR (CDCl₃, 300 MHz) δ 7.84 (d, 1H. J=5.4 Hz), 7.83 (d, 1H. J=5.4 Hz), 7.69 (d, 1H, J=5.4 Hz), 7.68 (d, 1H, J=5.4 Hz), 7.20 (d, 0.5H, J=5.2 Hz), 7.19 (d, 0.5H, J=5.2 Hz), 7.14 (m, 1H), 6.92 (d, 0.5H, J=5.1 Hz), 6.91 (d, 0.5H, J=5.1 Hz), 5.01 (s, 2H). HPLC (Condition A), Rt: 4.11 min (HPLC purity: 99%).

### Step b) Formation of 5-[(1,3-dioxo-1,3-dihydro-2H-isoindol-2-yl)methyl]thiophene-2-sulfonyl chloride

To a cold (−78° C.) solution of 2-(thien-2-ylmethyl)-1H-isoindole-1,3(2H)-dione (6.8 g, 27.9 mmol) in DCM (56 mL) obtained under step a) was added dropwise (in about 10 min) chlorosulfonic acid (16.2 g, 139.3 mmol, 9.3 mL, d: 1.74) diluted in DCM (9.3 mL). The reaction mixture was stirred for 2 h at −78° C., then for 1 h at −40° C. and overnight at rt. The resulting brown solution was poured on ice. The mixture was extracted with DCM (3× 200 mL), and the combined organic layers were washed with water (3×200 mL), dried over MgSO₄, filtered and concentrated to afford a yellowish oil. This crude product was purified by column chromatography over silica gel (AcOEt/c-Hex 1/4 to 1/3 to 1/2 in about 1 h) to give the title compound as a white solid (6.4 g, 67%). ¹H NMR (CDCl₃, 300 MHz) δ 7.89 (d, 1H. J=5.5 Hz), 7.87 (d, 1H. J=5.5 Hz), 7.76 (d, 1H, J=5.5 Hz), 7.75 (d, 1H, J=5.5 Hz), 7.71 (d, 1H, J=4.0 Hz), 7.18 (d, 1H, J=4.0 Hz), 5.05 (s, 2H). HPLC (Condition A), Rt: 4.6 min (HPLC purity: 94.8%).

### Step c) Formation of 5-[(1,3-dioxo-1,3-dihydro-2H-isoindol-2-yl)methyl]-N-dodecylthiophene-2-sulfonamide

To a solution of 5-[(1,3-dioxo-1,3-dihydro-2H-isoindol-2-yl)methyl]thiophene-2-sulfonyl chloride (2 g, 5.8 mmol) obtained under step b), DTEA (1.1 g, 8.8 mmol) in DCM (20 mL) was added dodecyl amine (1.4 g, 7.6 mmol) at rt and the reaction mixture was stirred for 2 h at rt. A 1 M aqueous solution of HCl (10 mL) was added and the aqueous layers were extracted with DCM (2×30 mL). The combined organic layers were dried over MgSO₄, filtered and concentrated to afford a yellowish oil. This crude product was purified by column chromatography over silica gel (AcOEt/c-Hex 1/4 to 4/1 in about 0.5 h) to give the title compound as a white solid (2.1 g, 73%). ¹H NMR (CD₃OD, 300 MHz) δ 7.91 (m, 2H), 7.85 (m, 2H), 7.43 (d, 1H, J=3.7 Hz), 7.17 (d, 1H, J=3.7 Hz), 5.05 (s, 2H), 2.90 (t, 2H, J=6.9 Hz), 1.50-1.38 (m, 2H), 1.35-1.16 (m, 18H), 0.86 (t, J=7.9 Hz, 3H). M⁻(LC/MS): 489.3; M⁺(LC/MS): 491.2. HPLC (Condition A), Rt: 6.64 min (HPLC purity: 96%).

### Step d) Deprotection of 5-[(1,3-dioxo-1,3-dihydro-2H-isoindol-2-yl)methyl]-N-dodecylthiophene-2-sulfonamide; formation of 5-(aminomethyl)-N-dodecylthiophene-2-sulfonamide

To a solution of 5-[(1,3-dioxo-1,3-dihydro-2H-isoindol-2-yl)methyl]-N-dodecylthiophene-2-sulfonamide obtained under step c) (2.0 g, 4.2 mmol) in EtOH (20 mL) was added hydrazine hydrate (0.61 mL, 633 mg, d: 1.030, 12.6 mmol). The resulting reaction mixture was stirred at reflux for 3 h and then cooled down to rt. The white precipitate was removed by filtration and the solvents were evaporated under vacuum. The residue was dissolved in DCM (20 mL) and the precipitate removed by filtration. The collected solvents were concentrated to afford of a colorless oil which turns solid on standing (1.5 g, 99%). ¹H NMR (DMSO-d₆, 300 MHz) δ 7.37 (m, 1H), 6.94 (m, 1H), 3.91 (s, 2H), 2.78 (m, 2H), 1.95-1.65 (m, 20H), 0.86 (t, J=7.6 Hz, 3H). M⁻(LC/MS (ESI)): 359.2; M⁺(LC/MS (ESI)): 361.2, HPLC (Condition A), Rt: 4.5 min (HPLC purity: 95%).

### Step e) Formation of N-dodecyl-5-({[4-(trifluoromethyl)benzyl]amino}methyl)thiophene-2-sulfonamide

To a solution of 5-(aminomethyl)-N-dodecylthiophene-2-sulfonamide obtained under step d) (797 mg, 2.2 mmol) and 4-trifluoromethyl-benzaldehyde (350 mg, 2.0 mmol) in DCE (50 mL) was added at once NaBH(OAc)₃ (596 mg, 2.8 mmol) and the resulting mixture was stirred overnight at rt. 30 mL of a saturated aqueous solution of NaHCO₃ were added to the reaction mixture, the aqueous layer was separated and washed with DCM (3×200 mL). The combined organic layers were dried over MgSO₄, filtered and concentrated to afford a yellowish oil. This crude product was purified by column chromatography over silica gel (AcOEt/c-Hex 1/4 to 1/2 in about 1 h) to give the title compound as a colorless oil (675 mg, 64%). ¹H NMR (CDCl₃, 300 MHz) δ 7.60 (m, 2H), 7.46 (m, 2H), 7.37 (d, 0.7H, J=8.0 Hz), 6.88 (d, 1H, J=3.8 Hz), 4.00 (s, 2H), 3.90 (s, 2H), 3.02 (m, 2H), 1.85-1.55 (m, 2H), 1.5 (m, 2H), 1.22 (s, 18H), 0.87 (t, 3H, 6.6 Hz). M⁻(LC/MS (ESI)): 517.2; M⁺(LC/MS (ESI)): 519.2. HPLC (Condition A), Rt: 5.27 min (HPLC purity: 97%).

### Step f) Formation of ethyl {({5-[(dodecylamino)sulfonyl]thien-2-yl}methyl)[4-(trifluoromethyl)benzyl]amino}(oxo)acetate

To a solution of N-dodecyl-5-({[4-(trifluoromethyl)benzyl]amino}methyl)thiophene-2-sulfonamide obtained under step e) (670 mg, 1.29 mmol) and TEA (2 eq., 261 mg, 2.5 mmol) in anhydrous THF (15 mL) at 0° C. under inert atmosphere, was added dropwise the chloro-oxo-acetic acid ethyl ester (1.5 eq., 265 mg, 1.9 mmol) diluted in THF (2 mL). The reaction mixture was stirred at RT for 3 h. The solvent was evaporated and DCM was added. The solution was washed with water (3×). The combined organic layers were dried over MgSO₄, filtered and concentrated to afford a yellowish oil. This crude product was purified by column chromatography over silica gel (AcOEt/c-Hex 1/7) to give the title compound as a colorless oil (360 mg, 45%). ¹H NMR (CDCl₃, 300 MHz) δ 7.66 (t, 2H, J=9.0 Hz), 7.42 (m, 2H), 7.37 (d, 0.7H, J=8.0 Hz), 6.87 (d, 0.3H, J=3.8 Hz), 6.86 (d, 0.7H, J=3.8 Hz), 4.60 (m, 2H), 4.52 (m, 2H), 4.36 (m, 2H), 3.02 (m, 2H), 1.50 (m, 3H), 1.40-1.20 (m, 21H), 0.86 (t, 3H, 6.6 Hz) M⁻(APCI): 617.2; M⁺(APCI): 619.2 HPLC (Condition A), Rt: 7.1 min (HPLC purity: 99.9%).

### Step g) Formation of {({5-[(dodecylamino)sulfonyl]-2-thienyl}methyl)[4-(trifluoromethyl)-benzyl]amino}(oxo)acetic acid

To a solution of ethyl {({5-[(dodecyliamino)sulfonyl]thien-2-yl}methyl)[4-(trifluoromethyl)benzyl]amino}(oxo)acetate obtained under step f) (356 mg, 0.58 mmol) in EtOH (1.5 mL) was added a aq. solution of NaOH (1.1 eq., 25 mg, 0.63 mmol) in H₂O (0.5 mL) and the resulting reaction mixture was stirred at rt for 1 h. The solvents were evaporated and the residue dissolved in EtOAc (20 mL) and washed with a 1N aqueous solution of HCl (5 mL). The aqueous layer was separated and washed with EtOAc (2×10 mL). The combined organic layers were dried over MgSO₄, filtered and concentrated to afford the title compound (1) as a foam (325 mg, 96%). ¹H NMR (CD₃OD, 300 MHz) δ 7.61 (m, 2H), 7.52 (m, 1H), 7.40 (m, 1H), 7.32 (m, 1H), 7.08 (m, 0.5H), 6.85 (m, 0.5H), 4.71 (m, 4H), 2.88 (m, 2H), 1.46 (m, 2H), 1.27 (m, 18H), 0.87 (t, J=8.1 Hz, 3H). M⁻(LC/MS (ESI)): 589.1; M⁺(LC/MS (ESI)): 591.3. HPLC (Condition A), Rt: 6.58 min (HPLC purity: 99.9%)

### Example 2

### [benzyl({5-[(dodecylamino)sulfonyl]-2-thienyl}methyl)amino](oxo)acetic acid (2)

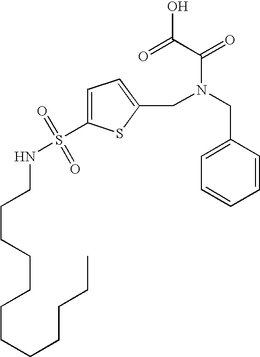

### Step a) Formation of the resin-bound amines of formula (D) (See Scheme 5), e.g. the resin-bound dodecylamine

The resin PS-MB-CHO HL (Argonaut Technologies Inc., 30 mg, 1.42 mmol/g, 0.0426 mmol, 100-200 mesh) was swelled in 1% HAc in DCE/TMOF (80/20) (1.0 mL) for 15 min at rt. Dodecylamine (24 mg, 0.128 mmol) and sodium triacetoxyborohydride (27 mg, 0.128 mmol) were added and the reaction mixture was shaken at rt for 14 h. The resin was washed successively with THF (1×15 min), MeOH (1×15 min), THF (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the resin-bound dodecylamine which was used directly in the next step.

### Step b) Formation of the resin-bound protected amines of formula (VII-1) (See Scheme 5, Method L), e.g. the resin-bound 5-[(1,3-dioxo-1,3-dihydro-2H-isoindol-2-yl)methyl]-N-dodecyl-thiophene-2-sulfonamide

The resin-bound dodecylamine (described in step a), 0.0426 mmol) was swelled in DCM (1.0 mL) for 15 min at rt. DIEA (33 mg, 0.256 mmol) and 5-[(1,3-dioxo-1,3-dihydro-2H-isoindol-2-yl)methyl]thiophene-2-sulfonyl chloride (44 mg, 0.128 mmol) were added and the resulting reaction mixture was shaken 14 h at rt. The resin was washed successively with NMP (1×15 min), MeOH (1×15 min), THF (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the title compound which was used directly in the next step.

### Step c) Phtalimide-deprotection of the resin-bound protected amines of formula (VII-1) (See Scheme 5); e.g formation of the resin-bound 5-(aminomethyl)-N-dodecylthiophene-2-sulfonamide

The resin-bound 5-[(1,3-dioxo-1,3-dihydro-2H-isoindol-2-yl)methyl]-N-dodecylthiophene-2-sulfonamide (described in step b), 0.0426 mmol) was treated with a 60% solution (v/v) hydrazine monohydrate in DMF (1.15 mL) and shaken 14 h at rt. The resin was washed successively with DMF (1×15 min), MeOH (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the title compound which was used directly in the next step.

### Step d) Formation of the resin-bound secondary amines of formula (III-1) (See Scheme 5, Method L), e.g. the resin-bound 5-[(benzylamino)methyl]-N-dodecylthiophene-2-sulfonamide

The resin-bound 5-(aminomethyl)-N-dodecylthiophene-2-sulfonamide (described in step c), 0.0426 mmol) was swelled in THF/TMOF 90/10 (1.0 mL) for 15 min at rt. Benzaldehyde (45 mg, 0.426 mmol) was added and the mixture was shaken 14 h at rt. The resin was washed with 10% TMOF in anhydrous THF (2×15 min, then 2×60 min), then with anhydrous THF (1×30 min). The resin was then poured in anhydrous THF (1.0 mL). Sodium triacetoxyborohydride (30 mg, 0.140 mmol) and acetic acid (9 μL) was added and the mixture was shaken 14 h at rt. The resin was washed successively with THF (1×15 min), MeOH (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the title compound which was used directly in the next step.

### Step e) Formation of the resin-bound oxamic ester of formula (I-1) (See Scheme 1), e.g. resin-bound ethyl [benzyl({5-[(dodecylamino)sulfonyl]thien-2-yl}methyl)amino]-(oxo)acetate

The resin-bound 5-[(benzylamino)methyl]-N-dodecylthiophene-2-sulfonamide (described in step d), 0.0426 mmol) was swelled in DCM (1.0 mL) for 15 min at 0° C. DTEA (28 mg, 0.213 mmol) and a solution of chloro-oxo-acetic acid ethyl ester (29 mg, 0.213 mmol) in DCM (0.2 mL) were added and the reaction mixture was shaken for 4 h at rt. The resin was washed successively with THF (1×15 min), MeOH (1×15 min), THF (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the title compound which was used directly in the next step.

### Step f) Formation of the resin-bound thienyl of formula (I-1) (See Scheme 1), e.g. resin-bound [benzyl({5-[(dodecylamino)sulfonyl]-2-thienyl}methyl)amino](oxo)acetic acid

The resin-bound ethyl [benzyl({5-[(dodecylamino)sulfonyl]thien-2-yl}methyl)amino](oxo)acetate (described in step e), 0.0426 mmol) was swelled in THF/H₂O 80/20 (0.5 mL) for 15 min at rt. Lithium hydroxide monohydrate (20 eq. 38 mg, 0.9 mmol) diluted in THF/H₂O 80/20 (0.8 mL) was added and the resulting reaction mixture was shaken for 14 h at rt. The resin was washed successively with THF (1×15 min), H₂O (1×15 min), MeOH (1×15 min), THF (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the title compound which was used directly in the next step.

### Step g) Cleavage of the Resin-Bound Thienyl of Formula (I-1); Formation of the Thienyl of Formula (I) (See Scheme 1), e.g. [benzyl({5-[(dodecylamino)sulfonyl]-2-thienyl}-methyl) amino](oxo)acetic acid

The resin-bound [benzyl({5-[(dodecylamino)sulfonyl]-2-thienyl}methyl)amino](oxo)acetic acid (described in step f), 0.0426 mmol) was poured in TFA/DCM 20/80 (2 mL) for 1 h at rt. The resin was filtered and the solvents were evaporated under vacuum to afford a colorless oil. The crude product was purified by reverse phase chromatography (Parrallel Flex) following a gradient of 30% 0.1% TFA in H₂O in 0.1% TFA in ACN up to 100% 0.1% TFA in ACN in 10 min, followed by elution with ACN (4 min.). The collected fractions were lyophilized to give the title compound (2) as a white gum (20 mg). M⁻(LC/MS (ESI)): 521.2; M⁺(LC/MS (ESI)): 523.0. HPLC (Condition A), Rt: 6.17 min (HPLC purity: 86.2%).

### Example 3

### [cyclopentyl({5-[(dodecylamino)sulfonyl]-2-thienyl}methyl)amino](oxo)acetic acid (3)

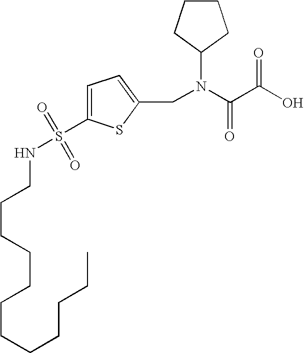

### Step a) Formation of the resin-bound 5-[(cyclopentylamino)methyl]-N-dodecylthiophene-2-sulfonamide

The resin-bound 5-(aminomethyl)-N-dodecylthiophene-2-sulfonamide (Example 2, step c), 0.23 mmol) was swelled in a 1% HAc in DMF mixture for 15 min at rt. Cyclopentanone (97 mg, 1.15 mmol) and sodium cyanoborohydride (144 mg, 2.3 mmol) were then added and the reaction mixture shaken for 14 h at rt. The resin was washed successively with DMF (1×15 min), MeOH (1×15 min), THF (1×15 min), MeOH (3×10 min), DMF (3×10 min), MeOH (1×5 min), THF (3×10 min), MeOH (1×5 min), DCM (3×10 min) and with Et₂O (1×10 min). The resin was then dried under vacuum to afford the title compound which was used directly in the next step.

Step b) Formation of the resin-bound ethyl [cyclopentyl({5-[(dodecylamino)sulfonyl]thien-2-yl}methyl)amino](oxo)acetate

The same procedure as employed in the preparation of Example 2, step e) but using resin-bound 5-[(cyclopentylamino)methyl]-N-dodecylthiophene-2-sulfonamide gave the title compound which was used directly in the next step.

### Step c) Cleavage of the resin bound ethyl [cyclopentyl({5-[(dodecylamino)sulfonyl]thien-2-yl}methyl)amino](oxo)acetate; formation of the ethyl [cyclopentyl({5-[(dodecylamino)sulfonyl]thien-2-yl}methyl)amino](oxo)acetate

The same procedure as employed in the preparation of Example 2, step g) but using resin-bound ethyl [cyclopentyl({5-[(dodecylamino)sulfonyl]thien-2-yl}methyl)amino](oxo) acetate (obtained under step b) gave a yellow oil. This crude product was purified by column chromatography over silica gel to give the title compound (11 mg, 10%). M⁻(LC/MS (ESI)): 527.2; M⁺(LC/MS (ESI)): 529.4. HPLC (Condition A), Rt: 6.94 min (HPLC purity: 91.0%).

Step d) Formation of [cyclopentyl({5-[(dodecylamino)sulfonyl]-2-thienyl}methyl)amino](oxo)acetic acid

The same procedure as employed in the preparation of Example 1, step g) but using ethyl [cyclopentyl({5-[(dodecylamino)sulfonyl]thien-2-yl}methyl)amino](oxo)acetate gave the title compound (3) as a colorless foam (96%). ¹H NMR (CD₃OD, 300 MHz) δ 7.25 (m, 1H), 7.0 (m, 1H), 4.64 (s, 1H), 4.30 (m, 1H), 2.76 (t, 2H, J=7.3 Hz), 1.81 (m, 2H), 1.79-1.41 (m, 8H), 1.29 (m, 19H), 0.91 (t, 3H, J=6.8 Hz). M⁻(LC/MS (ESI)): 499.2; M⁺(LC/MS (ESI)): 501.2. HPLC (Condition A), Rt: 6.09 min (HPLC purity: 78.7%).

### Example 4

### (({5-[(dodecylamino)sulfonyl]-2-thienyl}methyl){13-[hydroxy(oxido)amino]benzyl}amino)(oxo)acetic acid (4)

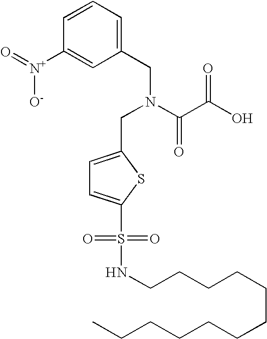

The same procedure as employed in the preparation of Example 2 using dodecylamine in step a) and 3-nitrobenzaldehyde in step d gave the title compound (4) as an orange oil (29 mg). M⁻(LC/MS (ESI)): 566.3; M⁺(LC/MS (ESI)): 568.2. HPLC (Condition A), Rt: 6.23 min (HPLC purity: 61.7%).

### Example 5

### [({5-[(dodecylamino)sulfonyl]-2-thienyl}methyl)(4-methoxybenzyl)amino]-(oxo)acetic acid (5)

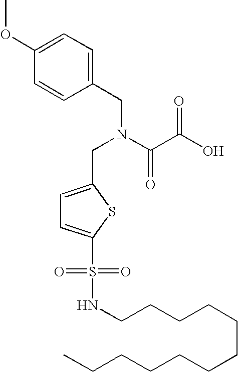

The same procedure as employed in the preparation of Example 2 using dodecylamine in step a) and p-anisaldehyde in step d gave the title compound (5) as a yellow oil (27 mg). M⁻(LC/MS (ESI)): 551.2; M⁺(LC/MS (ESI)): 553.4. HPLC (Condition A), Rt: 6.26 min (HPLC purity: 73.3%).

### Example 6

### [({5-[(dodecylamino)sulfonyl]-2-thienyl}methyl)(2-fluorobenzyl)amino]-(oxo)acetic acid (6)

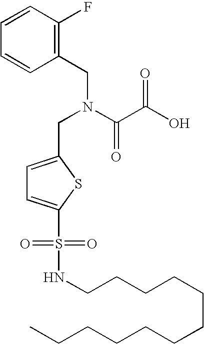

The same procedure as employed in the preparation of Example 2 using dodecylamine in step a) and 2-fluorobenzaldehyde in step d) gave the title compound (6) as a yellow solid (28 mg). M⁻(LC/MS (ESI)): 539.1; M⁺(LC/MS (ESI)): 541.2. HPLC (Condition A), Rt: 6.33 min (HPLC purity: 70%).

### Example 7

### {({5-[(dodecylamino)sulfonyl]-2-thienyl}methyl) [4-(methylsulfonyl)-benzyl]amino}(oxo)acetic acid (7)

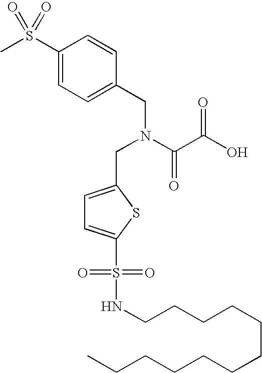

The same procedure as employed in the preparation of Example 2 using dodecylamine in step a) and 4-(methylsulfonyl)benzaldehyde in step d) gave the title compound (7) as a yellow oil (36 mg). M⁻(LC/MS (ESI)): 599.2; M⁺(LC/MS (ESI)): 601.3. HPLC (Condition A), Rt: 5.81 min (HPLC purity: 69.4%).

### Example 8

### [({5-[(dodecylamino)sulfonyl]-2-thienyl}methyl)(4-phenoxybenzyl)amino]-(oxo)acetic acid (8)

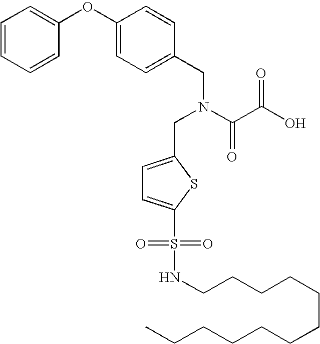

The same procedure as employed in the preparation of Example 2 using dodecylamine in step a) and 4-phenoxybenzaldehyde in step d) gave the title compound (8) as a yellow oil (33 mg). M⁻(LC/MS (ESI)): 613.2; M⁺(LC/MS (ESI)): 615.0. HPLC (Condition A), Rt: 6.78 min (HPLC purity: 68.5%).

### Example 9

### 4-{[(carboxycarbonyl)({5-[(dodecylamino)sulfonyl]-2-thienyl}methyl)-amino]methyl}benzoic acid (9)

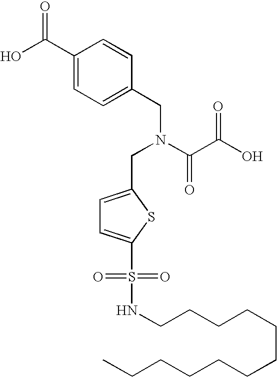

The same procedure as employed in the preparation of Example 2 using dodecylamine in step a) and methyl 4-formylbenzoate in step d) gave the title compound (9) as a yellow oil (5 mg). M⁻(LC/MS (ESI)): 565.3; M⁺(LC/MS (ESI)): 567.3. HPLC (Condition A), Rt: 5.43 min (HPLC purity: 99.9%).

### Example 10

### (({5-[(dodecylamino)sulfonyl]-2-thienyl}methyl){[6-(trifluoromethyl)-3-pyridinyl]methyl}amino)(oxo)acetic acid (10)

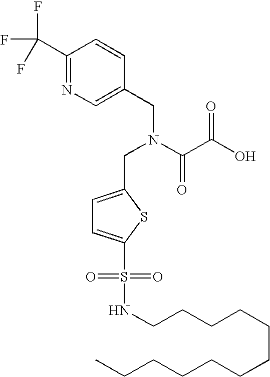

The same procedure as employed in the preparation of Example 2 using dodecylamine in step a) and 6-(trifluoromethyl)pyridine-3-carboxaldehyde in step d) gave the title compound (10) as an orange oil (30 mg). M⁻(LC/MS (ESI)): 590.3; M⁺(LC/MS (ESI)): 592.2. HPLC (Condition A), Rt: 6.25 min (HPLC purity: 61.7%).

### Example 11

### {({5-[(dodecylamino)sulfonyl]-2-thienyl}methyl) [3-(trifluoromethyl)-benzyl]amino}(oxo)acetic acid (11)

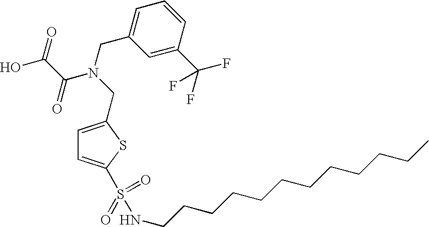

The same procedure as employed in the preparation of Example 2 using dodecylamine in step a) and 3-(trifluoromethyl)benzaldehyde in step d) gave the title compound (11) as a yellow oil (19 mg). M⁻(LC/MS (ESI)): 589.3; M⁺(LC/MS (ESI)): 591.3. HPLC (Condition A), Rt: 6.43 min (HPLC purity: 81.5%).

### Example 12

### [(3-chlorobenzyl)({5-[(dodecylamino)sulfonyl]-2-thienyl}methyl)amino]-(oxo)acetic acid (12)

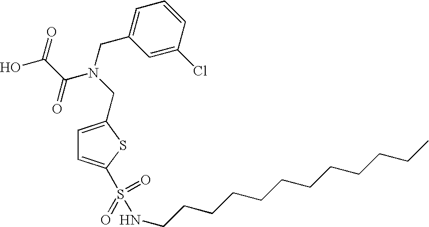

The same procedure as employed in the preparation of Example 2 using dodecylamine in step a) and 3-chlorobenzaldehyde in step d) gave the title compound (12) as a yellow oil (21 mg). M⁻(LC/MS (ESI)): 556; M⁺(LC/MS (ESI)): 558. HPLC (Condition A), Rt: 6.32 min (HPLC purity: 81.9%).

### Example 13

### {[(5-{[(3,3-diphenylpropyl)amino]sulfonyl}-2-thienyl)methyl][3-(trifluoro-methyl)benzyl]amino}(oxo)acetic acid (13)

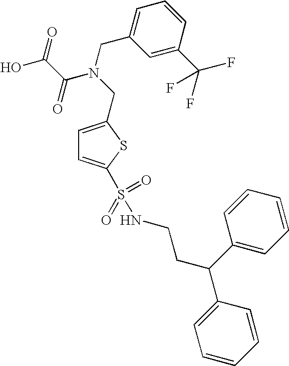

The same procedure as employed in the preparation of Example 2 using 3,3-diphenyl-propylamine in step a) and 3-(trifluoromethyl)benzaldehyde in step d) gave the title compound (13) as a yellow oil (17 mg). M⁻(LC/MS (ESI)): 615.3; M⁺(LC/MS (ESI)): 617.3. HPLC (Condition A), Rt: 5.12 min (HPLC purity: 75.7%).

### Example 14

### {(3-chlorobenzyl) [(5-{[(3,3-diphenylpropyl)amino]sulfonyl}-2-thienyl)-methyl]amino}(oxo)acetic acid (14)

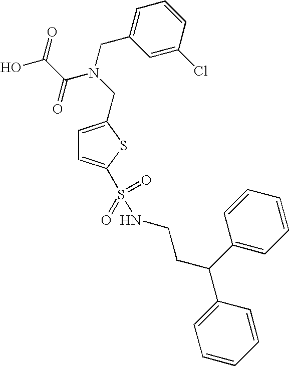

The same procedure as employed in the preparation of Example 2 using 3,3-diphenyl-propylamine in step a) and 3-chlorobenzaldehyde in step d) gave the title compound (14) as a yellow oil (15 mg). M⁻(LC/MS (ESI)): 582.5; M⁺(LC/MS (ESI)): 585.1. HPLC (Condition A), Rt: 5.01 min (HPLC purity: 72.1%).

### Example 15

### oxo{{[5-({[2-(4-phenoxyphenyl)ethyl]amino}sulfonyl)-2-thienyl]methyl}[3-(trifluoromethyl)benzyl]amino}acetic acid (15)

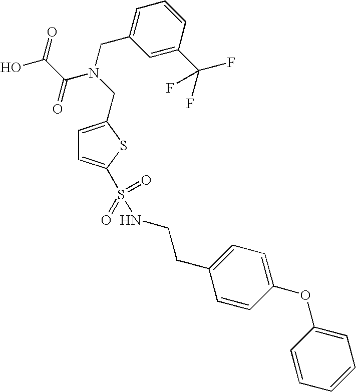

The same procedure as employed in the preparation of Example 2 using 4-phenoxyphenethylamine in step a) and 3-(trifluoromethyl)benzaldehyde in step d) gave the title compound (15) as a yellow oil (22 mg). M⁻(LC/MS (ESI)): 617.0; M⁺(LC/MS (ESI)): 619.0. HPLC (Condition A), Rt: 5.15 min (HPLC purity: 77.1%).

### Example 16

### ((3-chlorobenzyl){[5-({[2-(4-phenoxyphenyl)ethyl]amino}sulfonyl)-2-thienyl]methyl}amino)(oxo)acetic acid (17)

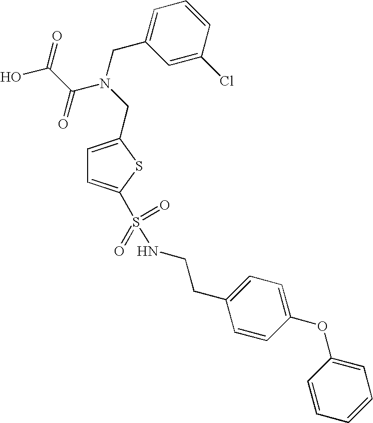

The same procedure as employed in the preparation of Example 2 using 4-phenoxy-phenethylamine in step a) and 3-chlorobenzaldehyde in step d) gave the title compound (16) as a yellow oil (20 mg). M⁻(LC/MS (ESI)): 584; M⁺(LC/MS (ESI)): 586. HPLC (Condition A), Rt: 5.0 min (HPLC purity: 79%).

### Example 17

### {[(5-{[(2-[1,1′-biphenyl]-4-ylethyl)amino]sulfonyl}-2-thienyl)methyl][3-(trifluoromethyl)benzyl]amino}(oxo)acetic acid (17)

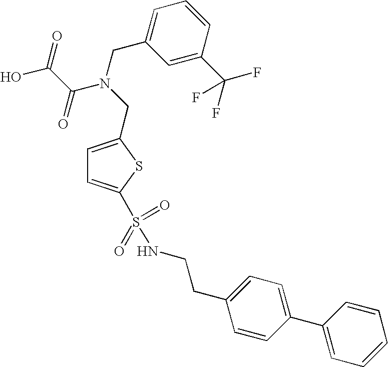

The same procedure as employed in the preparation of Example 2 using 2-(4-biphenyl)-ethylamine in step a) and 3-(trifluoromethyl)benzaldehyde in step d) gave the title compound (17) as a yellow oil (20 mg). M⁻(LC/MS (ESI)): 601.2; M⁺(LC/MS (ESI)): 603.0. HPLC (Condition A), Rt: 5.13 min (HPLC purity: 71.4%).

### Example 18

### Formation of {({5-[(hexylamino)sulfonyl]-2-thienyl}methyl) [3-(trifluoro methyl)benzyl]amino}(oxo)acetic acid (18)

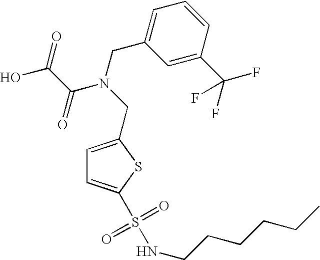

The same procedure as employed in the preparation of Example 2 using hexylamine in step a) and 3-trifluoromethyl-benzaldehyde in step d) gave the title compound (18) as a yellow oil (17 mg). M⁻(LC/MS (ESI)): 505.1; M⁺(LC/MS (ESI)): 507. HPLC (Condition A), Rt: 4.9 min (HPLC purity: 81.8%).

### Example 19

### Formation of [[(5-{[(2-biphenyl-4-ylethyl)amino]sulfonyl}-2-thienyl)methyl](pyridin-4-ylmethyl)amino](oxo)acetic acid (19)

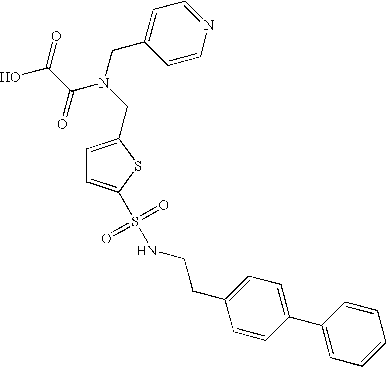

The same procedure as employed in the preparation of Example 2 using 2-biphenyl-4-yl-ethylamine in step a) and pyridine-4-carbaldehyde in step d) gave the title compound (19) as a yellow solid (1 mg). M⁻(LC/MS (ESI)): 534; M⁺(LC/MS (ESI)): 536.2. HPLC (Condition A), Rt: 3.3 min (HPLC purity: 95.5%).

### Example 20

### Formation of 4-({[5-({(carboxycarbonyl) [3-(trifluoromethyl)benzyl]amino}methyl)-2-thienyl]sulfonyl}amino)butanoic acid (20)

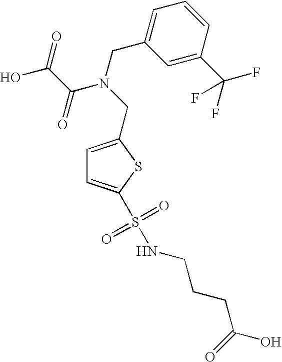

The same procedure as employed in the preparation of Example 2 using 4-amino-butyric acid t-butyl ester in step a) and 3-trifluoromethyl-benzaldehyde in step d) gave the title compound (20) as a yellow oil (20 mg). M⁻(LC/MS (ESI)): 507; M⁺(LC/MS (ESI)): 509.1. HPLC (Condition A), Rt: 3.24 min (HPLC purity: 77.1%).

### Example 21

### Formation of 4-{[(5-{[(carboxycarbonyl)(3-chlorobenzyl)amino]methyl}-2-thienyl)sulfonyl]amino}butanoic acid (21)

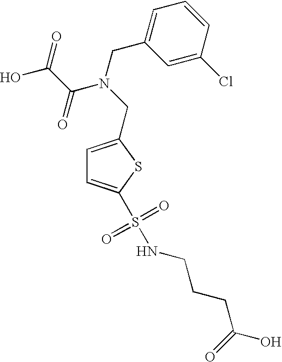

The same procedure as employed in the preparation of Example 2 using 4-amino-butyric acid t-butyl ester in step a) and 3-chloro-benzaldehyde in step d) gave the title compound (21) as a yellow oil (14 mg). M⁻(LC/MS (ESI)): 473; M⁺(LC/MS (ESI)): 475. HPLC (Condition A), Rt: 2.95 min (HPLC purity: 74.5%).

### Example 22

### Formation of {{[5-({[2-(4-chlorophenyl)propyl]amino}sulfonyl)-2-thienyl]methyl}[3-(trifluoromethyl)benzyl]amino}(oxo)acetic acid (22)

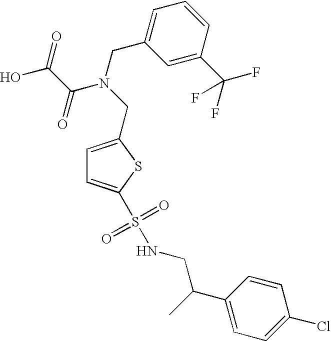

The same procedure as employed in the preparation of Example 2 using 2-(4-chlorophenyl)-propylamine in step a) and 3-trifluoromethyl-benzaldehyde in step d) gave the title compound (22) as a yellow oil (18 mg). M⁻(LC/MS (ESI)): 573; M⁺(LC/MS (ESI)): 575. HPLC (Condition A), Rt: 4.96 min (HPLC purity: 80.3%).

### Example 23

### Formation of [{[5-({[2-(4-chlorophenyl)propyl]amino}sulfonyl)-2-thienyl]methyl}(pyridin-4-ylmethyl)amino](oxo)acetic acid (23)

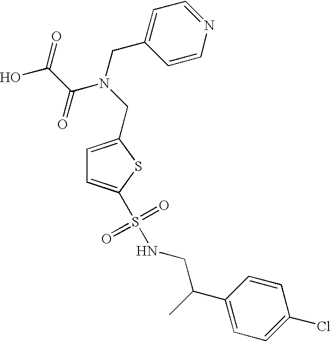

The same procedure as employed in the preparation of Example 2 using 2-(4-chlorophenyl)-propylamine in step a) and pyridine-4-carbaldehyde in step d) gave the title compound as a yellow powder (1 mg). M⁻(LC/MS (ESI)): 506; M⁺(LC/MS (ESI)): 508. HPLC (Condition A), Rt: 2.97 min (HPLC purity: 78.8%).

### Example 24

### Formation of ((3-chlorobenzyl){[5-({[2-(4-chlorophenyl)propyl]amino}sulfonyl)-2-thienyl]methyl}amino)(oxo)acetic acid (24)

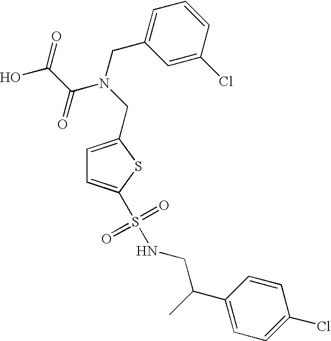

The same procedure as employed in the preparation of Example 2 using 2-(4-chlorophenyl)-propylamine in step a) and 3-chloro-benzaldehyde in step d) gave the title compound (24) as a yellow oil (14 mg). M⁻(LC/MS (ESI)): 538.9; M⁺(LC/MS (ESI)): 540. HPLC (Condition A), Rt: 4.85 min (HPLC purity: 77.9%).

### Example 25

### Formation of {[(5-{[(2-ethylhexyl)amino]sulfonyl}-2-thienyl)methyl][3-(trifluoromethyl)benzyl]amino}(oxo)acetic acid (25)

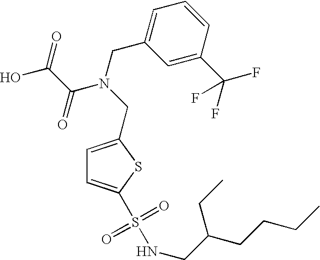

The same procedure as employed in the preparation of Example 2 using 2-ethyl-hexylamine in step a) and 3-trifluoromethyl-benzaldehyde in step d) gave the title compound (25) as a yellow oil (7 mg). M⁻(LC/MS (ESI)): 533.2; M⁺(LC/MS (ESI)): 535.3. HPLC (Condition A), Rt: 5.37 min (HPLC purity: 98%).

### Example 26

### Formation of [[(5-{[(2-ethylhexyl)amino]sulfonyl}-2-thienyl)methyl](pyridin-4-ylmethyl)amino](oxo)acetic acid (26)

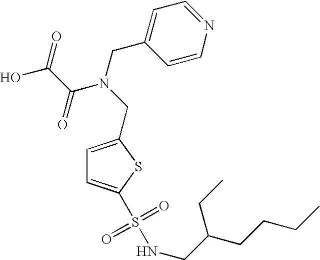

The same procedure as employed in the preparation of Example 2 using 2-ethyl-hexylamine in step a) and pyridine-4-carbaldehyde in step d) gave the title compound (26 as a yellow powder (2 mg). M⁻(LC/MS (ESI)): 466.1; M⁺(LC/MS (ESI)): 468.5. HPLC (Condition A), Rt: 3.18 min (HPLC purity: 67%).

### Example 27

### Formation of {(3-chlorobenzyl) [(5-{[(2-ethylhexyl)amino]sulfonyl}-2-thienyl)methyl]amino}(oxo)acetic acid (27)

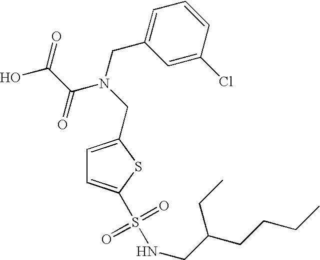

The same procedure as employed in the preparation of Example 2 using 2-ethyl-hexylamine in step a) and 3-chloro-benzaldehyde in step d) gave the title compound (27) as a yellow oil (17 mg). M⁻(LC/MS (ESI)): 499.1; M⁺(LC/MS (ESI)): 501. HPLC (Condition A), Rt: 5.21 min (HPLC purity: 82%).

### Example 28

### Formation of ((3-chlorobenzyl){[5-({[2-({[5-(dimethylamino)-1-naphthyl]sulfonyl}amino)ethyl]amino}sulfonyl)-2-thienyl]methyl}amino)(oxo)acetic acid (28)

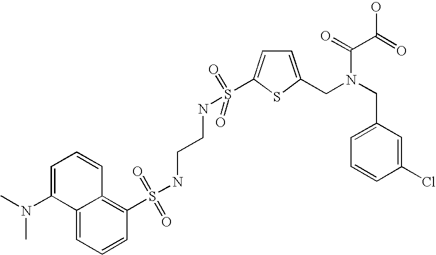

The same procedure as employed in the preparation of Example 1 using dansyl ethylenediamine in step a) and 3-chloro-benzaldehyde in step e) gave the hydrochloride salt of the title compound (28) as a yellow powder (15 mg). M⁻(LC/MS (ESI)): 665.5; M⁺(LC/MS (ESI)): 663.0 HPLC (Condition A), Rt: 2.83 min (HPLC purity: 95.8%).

### Example 29

### Biological Assays

The compounds of the present invention may be subjected to the following assays:

### a) In Vitro Testing:

The anti-bacterial activity of compounds of the invention can be evaluated by measuring the minimum inhibitory concentration (MIC) of the compounds of the invention. The is MIC₅₀ values can be established with and without the presence of 10% mouse serum for Mycobacterium tuberculosis H37Rv, according to the National Committee on Clinical Laboratory Standards (NCCLS) guidelines.

The compounds of the inventions have been tested in the following enzymatic assay:

Five μl of diluted compound or vehicle (100% DMSO) was distributed to a 96 well plate. 55 μl of DiFMUP 72.7 μM diluted in MPTPB buffer (20 mM Bis Tris HCl pH 6.6, 0.1% Brij 35, 1 mM DL-Dithiothreitol) was added, followed by 40 μl of recombinant MPTPB enzyme (50 ng/ml) diluted in MPTPB buffer in order to start the reaction. After 45 minutes incubation at room temperature fluorescence intensity was measured on a Perkin-Elmer Fusion spectrofluorimeter (excitation at 355 nm, emission at 460 nm, for 0.2 s).

Examples of inhibitory activities for compounds of the invention are set out in Table I below.

TABLE I
IC₅₀ values of thiazole derivatives against MPTPB

| | MPTPB |
|---|---|
| Example No | IC₅₀ (μM) |
| 6 | 0.74 |
| 14 | 0.79 |
| 24 | 0.77 |

### b) Animal Models

The guinea pig can be used for in the evaluation of M. tuberculosis load and activity as used since a long time (O'Grady et al., 1963, Adv. Tuberc. Res., 12, 150-190). However, the murine model such as described in Lecoeur et al., 1989, Clin. Exp. Immunol., 76, 458-462 is currently more used for the study of anti tuberculosis effects.

c) Other tests

Other tests for the observation of the growth of M. tuberculosis can be used such as the observation of the production of radioactive carbon dioxide in the BACTEC460 system such as described in Middlebrook et al., 1977, Ann. Rev. Respir. Dis., 115, 1066-1069 or of oxygen in the Mycobacterium growth indicator tube such as described in Pfyffer et al., 1997, J. Clin. Microbiol., 35, 364-368. The growth of M. tuberculosis can be also observed through the monitoring of the bioluminescence from Luciferase enzyme that is transducted into M. tuberculosis by a specifically-engineered virus (Hickey et al., 1996, Antimicrob. Agents Chemother., 32, 400-407).

### Example 30

### Preparation of a Pharmaceutical Formulation

### Formulation 1—Tablets

A compound of Formula (I) is admixed as a dry powder with a dry gelatin binder in an approximate 1:2 weight ration. A minor amount of magnesium stearate is added as a lubricant. The mixture is formed into 240-270 mg tablets (80-90 mg) of active sulfonamide compound per tablet) in a tablet press.

### Formulation 2—Capsules

A compound of Formula (I) is admixed as a dry powder with a starch diluent in an approximate 1:1 weight ratio. The mixture is filled into 250 mg capsules (125 mg of active sulfonamide compound per capsule).

### Formulation 3—Liquid

A compound of Formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 ml.

### Formulation 4—Tablets

A compound of Formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active sulfonamide compound) in a tablet press.

### Formulation 5—Injection

A compound of Formula (I) is dissolved in a buffered sterile saline injectable aqueous medium to a concentration of approximately 5 mg/ml.

1-23. (canceled)

24. A method of treating a bacterial infection comprising the administration, to an individual in need of treatment, a composition comprising a sulfonamide derivative according to Formula (I), 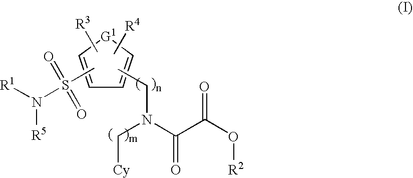
wherein:
G¹ is selected from —CR⁶R⁷—, —O—, —S— or —N(R⁸);
R¹ is selected from H, C₁-C₆ alkyl, C₇-C₁₂-alkyl, C₂-C₆ alkenyl, C₂-C₆ alkynyl, aryl C₁-C₆ alkyl, heteroaryl C₁-C₆ alkyl, C₃-C₈ cycloalkyl C₁-C₆ alkyl or heteroaryl C₁-C₆ alkyl;
R² is selected from H, C₁-C₆ alkyl, C₂-C₆ alkenyl, C₂-C₆ alkynyl or C₃-C₈ cycloalkyl;
R³ and R⁴ are independently selected from H, C₁-C₆ alkyl or halogen;
R⁵ is selected from H, C₁-C₆ alkyl, C₂-C₆ alkenyl, C₂-C₆ alkynyl or C₃-C₈ cycloalkyl;
R⁶, R⁷ and R⁸ are independently selected from H, C₁-C₆ alkyl, C₂-C₆ alkenyl or C₂-C₆ alkynyl;
Cy is selected from aryl, heteroaryl, C₃-C₈ cycloalkyl or heterocycloalkyl; and
m and n are integers independently selected from 0, 1, 2, 3 or 4;
or geometrical isomers, enantiomers, diastereomers, racemates, or pharmaceutically acceptable salts thereof.

25. The method according to claim 24, wherein G¹ is —S—.

26. The method according to claim 24, wherein R¹ is selected from C₁-C₆ alkyl, C₇-C₁₂ alkyl, C₂-C₆ alkenyl or C₂-C₆ alkynyl.

27. The method according to claim 24, wherein R¹ is selected from optionally substituted aryl C₁-C₆ alkyl or optionally substituted heteroaryl C₁-C₆ alkyl.

28. The method according to claim 24, wherein R² is H.

29. The method according to claim 24, wherein R³ is H.

30. The method according to claim 24, wherein R⁴ is H.

31. The method according to claim 24, wherein R⁵ is H.

32. The method according to claim 24, wherein R⁶ is H.

33. The method according to claim 24, wherein R⁷ is H.

34. The method according to claim 24, wherein m is 0.

35. The method according to claim 24, wherein m is 1.

36. The method according to claim 24, wherein n is 1.

37. The method according to claim 24, wherein R², R³, R⁴, R⁵, R⁶ and R⁷ are H; m is selected from 0 and 1; n is 1; G¹ is S; and R¹ and Cy are as described in claim 24.

38. The method according to claim 24, wherein the compound of Formula (J) is selected from:
{({5-[(dodecylamino)sulfonyl]-2-thienyl}methyl)[4-(trifluoromethyl)benzyl]amino}(oxo)acetic acid;
[benzyl({5-[(dodecylamino)sulfonyl]-2-thienyl}methyl)amino](oxo)acetic acid;
[cyclopentyl({5-[(dodecylamino)sulfonyl]-2-thienyl}methyl)amino](oxo)acetic acid;
[({5-[(dodecylamino)sulfonyl]-2-thienyl}methyl)(3-nitrobenzyl)amino](oxo)acetic acid;
[({5-[(dodecylamino)sulfonyl]-2-thienyl}methyl)(4-methoxybenzyl)amino](oxo) acetic acid;
[({5-[(dodecylamino)sulfonyl]-2-thienyl}methyl)(2-fluorobenzyl)amino](oxo)acetic acid;
{({5-[(dodecylamino)sulfonyl]-2-thienyl}methyl)[4-(methylsulfonyl)benzyl]amino}(oxo)acetic acid;
[({5-[(dodecylamino)sulfonyl]-2-thienyl}methyl)(4-phenoxybenzyl)amino](oxo) acetic acid;
4-{[(carboxycarbonyl)({5-[(dodecylamino)sulfonyl]-2-thienyl}methyl)amino]methyl}benzoic acid;
(({5-[(dodecylamino)sulfonyl]-2-thienyl}methyl) {[6-(trifluoromethyl)pyridin-3-yl]methyl}amino)(oxo)acetic acid;
{({5-[(dodecylamino)sulfonyl]-2-thienyl}methyl) [3-(trifluoromethyl)benzyl]amino}(oxo)acetic acid;
[(3-chlorobenzyl)({5-[(dodecylamino)sulfonyl]-2-thienyl}methyl)amino](oxo)acetic acid;
{[(5-{[(3,3-diphenylpropyl)amino]sulfonyl}-2-thienyl)methyl][3-(trifluoromethyl)benzyl]amino}(oxo)acetic acid;
{(3-chlorobenzyl)[(5-{[(3,3-diphenylpropyl)amino]sulfonyl}-2-thienyl)methyl]amino}(oxo)acetic acid;
oxo {{[5-({[2-(4-phenoxyphenyl)ethyl]amino}sulfonyl)-2-thienyl]methyl}[3-(trifluoromethyl)benzyl]amino}acetic acid;
((3-chlorobenzyl) {[5-({[2-(4-phenoxyphenyl)ethyl]amino}sulfonyl)-2-thienyl]methyl}amino)(oxo)acetic acid;
{[(5-{[(2-biphethyl-4-ylethyl)amino]sulfonyl}-2-thienyl)methyl][3-(trifluoromethyl)benzyl]amino}(oxo)acetic acid;
{({5-[(hexylamino)sulfonyl]-2-thienyl}methyl) [3-(trifluoromethyl)benzyl]amino}(oxo)acetic acid;
{({5-[(hexylamino)sulfonyl]-2-thienyl}methyl) [3-(trifluoromethyl)benzyl]amino}(oxo)acetic acid;
[[(5-{[(2-biphenyl-4-ylethyl)amino]sulfonyl}-2-thienyl)methyl](pyridin-4-ylmethyl)amino](oxo)acetic acid;
4-({[5-({(carboxycarbonyl)[3-(trifluoromethyl)benzyl]amino}methyl)-2-thienyl]sulfonyl}amino)butanoic acid;
4-{[(5-{[(carboxycarbonyl)(3-chlorobenzyl)amino]methyl}-2-thienyl)sulfonyl]amino}butanoic acid;
{{[5-({[2-(4-chlorophenyl)propyl]amino}sulfonyl)-2-thienyl]methyl}[3-(trifluoromethyl)benzyl]amino}(oxo)acetic acid;
[{[5-({[2-(4-chlorophenyl)propyl]amino}sulfonyl)-2-thienyl]methyl}(pyridin-4-yl methyl)amino](oxo)acetic acid;
((3-chlorobenzyl) {[5-({[2-(4-chlorophenyl)propyl]amino}sulfonyl)-2-thienyl]methyl}amino)(oxo)acetic acid;
{[(5-{[(2-ethylhexyl)amino]sulfonyl}-2-thienyl)methyl][3-(trifluoromethyl)benzyl]amino}(oxo)acetic acid;
[[(5-{[(2-ethylhexyl)amino]sulfonyl}-2-thienyl)methyl](pyridin-4-ylmethyl)amino](oxo)acetic acid;
{(3-chlorobenzyl) [(5-{[(2-ethylhexyl)amino]sulfonyl}-2-thienyl)methyl]amino}(oxo)acetic acid; or
((3-chlorobenzyl) {[5-({[2-({[5-(dimethylamino)-1-naphthyl]sulfonyl}amino)ethyl]amino}sulfonyl)-2-thienyl]methyl}amino)(oxo)acetic acid.

39. A method of inhibiting the activity of a Mycobacterium tuberculosis Protein Tyrosine Phosphatase (MPTP) comprising contacting M. tuberculosis with a compound Of Formula (I) 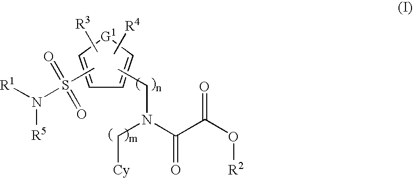
wherein:
G¹ is selected from —CR⁶R⁷—, —O—, —S— or —N(R⁸);
R¹ is selected from H, C₁-C₆ alkyl, C₇-C₁₂-alkyl, C₂-C₆ alkenyl, C₂-C₆ alkynyl, aryl C₁-C₆ alkyl, heteroaryl C₁-C₆ alkyl, C₃-C₈ cycloalkyl C₁-C₆ alkyl or heteroaryl C₁-C₆ alkyl;
R² is selected from H, C₁-C₆ alkyl, C₂-C₆ alkenyl, C₂-C₆ alkynyl or C₃-C₈ cycloalkyl;
R³ and R⁴ are independently selected from H, C₁-C₆ allyl or halogen;
R⁵ is selected from H, C₁-C₆ alkyl, C₂-C₆ alkenyl, C₂-C₆ alkynyl or C₃-C₈ cycloalkyl;
R⁶, R⁷ and R⁸ are independently selected from H, C₁-C₆ alkyl, C₂-C₆ alkenyl or C₂-C₆ alkynyl;
Cy is selected from aryl, heteroaryl, C₃-C₈ cycloalkyl or heterocycloalkyl; and
m and n are integers independently selected from 0, 1, 2, 3 and 4;
or geometrical isomers, enantiomers, diastereomers, racemates, or pharmaceutically acceptable salts thereof.

40. A compound selected from:
[[(5-{[(2-biphenyl-4-ylethyl)amino]sulfonyl}-2-thienyl)methyl](pyridin-4-ylmethyl)amino](oxo)acetic acid:
4-({[5-({(carboxycarbonyl) [3-(trifluoromethyl)benzyl]amino}methyl)-2-thienyl]sulfonyl}amino)butanoic acid;
4-{[(5-{[(carboxycarbonyl)(3-chlorobenzyl)amino]methyl}-2-thienyl)sulfonyl]amino}butanoic acid;
{{[5-({[2-(4-chlorophenyl)propyl]amino}sulfonyl)-2-thienyl]methyl}[3-(trifluoromethyl)benzyl]amino}(oxo)acetic acid;
[{[5-({[2-(4-chlorophenyl)propyl]amino}sulfonyl)-2-thienyl]methyl}(pyridin-4-ylmethyl)amino](oxo)acetic acid;
((3-chlorobenzyl){[5-({[2-(4-chlorophenyl)propyl]amino}sulfonyl)-2-thienyl]methyl}amino)(oxo)acetic acid;
{[(5-{[(2-ethylhexyl)amino]sulfonyl}-2-thienyl)methyl][3-(trifluoromethyl)benzyl]amino}(oxo)acetic acid;
[[(5-{[(2-ethylhexyl)amino]sulfonyl}-2-thienyl)methyl](pyridin-4-ylmethyl)amino](oxo)acetic acid;
{(3-chlorobenzyl)[(5-{[(2-ethylhexyl)amino]sulfonyl}-2-thienyl)methyl]amino}(oxo)acetic acid; or
((3-chlorobenzyl) {[5-({[2-({[5-(dimethylamino)-1-naphthyl]sulfonyl}amino)ethyl]amino}sulfonyl)-2-thienyl]methyl}amino)(oxo)acetic acid.

41. A pharmaceutical composition comprising a compound according to claim 40 and a pharmaceutically acceptable carrier, diluent or excipient thereof.

42. A process for the preparation of a compound comprising reacting an amine of Formula (II) with a carboxylic acid derivative of Formula LG₂-CO—CO—R² 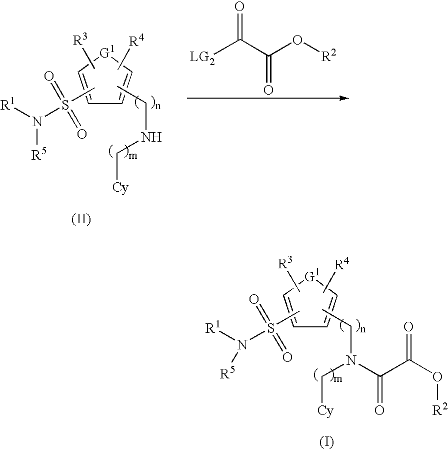
wherein:
R¹, R², R³, R⁴, R⁶, R⁷, m, G¹ and Cy are as described in claim 24, n is 1 and LG₂ is a leaving group selected from Cl, N-hydroxy succinimide or benzotriazol-1-yl.

43. A derivative of Formula (II) selected from:
Ethyl {({5-[(hexylamino)sulfonyl]-2-thienyl}methyl) [3 (trifluoromethyl)benzyl]amino}(oxo)acetate;
Ethyl [[(5-{[(2-biphenyl-4-ylethyl)amino]sulfonyl}-2-thienyl)methyl](pyridin-4-ylmethyl)amino](oxo)acetate;
4-({[5-({[ethoxy(oxo)acetyl][3-(trifluoromethyl)benzyl]amino}methyl)-2-thienyl]sulfonyl}amino)butanoic acid;
4-({[5-({(3-chlorobenzyl) [ethoxy(oxo)acetyl]amino}methyl)-2-thienyl]sulfonyl}amino)butanoic acid;
Ethyl {{[5-({[2-(4-chlorophenyl)propyl]amino}sulfonyl)-2-thienyl]methyl}[3-(trifluoromethyl)benzyl]amino}(oxo)acetate;
Ethyl [{[5-({[2-(4-chlorophenyl)propyl]amino}sulfonyl)-2-thienyl]methyl}(pyridin-4-ylmethyl)amino](oxo)acetate;
Ethyl ((3-chlorobenzyl){[5-({[2-(4-chlorophenyl)propyl]amino}sulfonyl)-2-thienyl]methyl}amino)(oxo)acetate:
Ethyl {[(5-{[(2-ethylhexyl)amino]sulfonyl}-2-thienyl)methyl][3-(trifluoromethyl)benzyl]amino}(oxo)acetate:
Ethyl [[(5-{[(2-ethylhexyl)amino]sulfonyl}-2-thienyl)methyl](pyridin-4-ylmethyl)amino](oxo)acetate;
Ethyl {(3-chlorobenzyl)[(5-{[(2-ethylhexyl)amino]sulfonyl}-2-thienyl)methyl]amino}(oxo)acetate; or
Ethyl ((3-chlorobenzyl) {[5-({[2-({[5-(dimethylamino)-1-naphthyl]sulfonyl}amino)ethyl]amino}sulfonyl)-2-thienyl]methyl}amino)(oxo)acetate.
